SEQUENCE LISTING (14 sequences)

SEQ ID NO: 1 - DNA, 5433 bp, Human
  CDS (1)...(5139)
  ref <301>: AUTHORS: Ryan,M.C., Tizard,R., VanDevanter,D.R. and Carter,W.G.
  ref <302>: TITLE: Cloning of the LamA3 gene encoding the alpha 3 chain of the adhesive ligand epiligrin. Expression in wound repair
  ref <303>: JOURNAL: JOURNAL   J. Biol. Chem.
  ref <304>: VOLUME: 269
  ref <305>: ISSUE: 36
  ref <306>: PAGES: 22779-22787
  ref <307>: DATE: 1994
atgggatggctgtggatctttggggcagccctggggcagtgtctgggctacagttcacagcagcaaagggtgccatttcttcagcctcccggtcaaagtcaactgcaagcgagttatgtggagtttagacccagccagggttgtagccctggatactatcgggatcataaaggcttgtataccggacggtgtgttccctgcaattgcaacggacattcaaatcaatgccaggatggctcaggcatatgtgttaactgtcagcacaacaccgcgggagagcactgtgaacgctgccaggagggctactatggcaacgccgtccacggatcctgcagggcctgcccatgtcctcacactaacagctttgccactggctgtgtggtgaatgggggagacgtgcggtgctcctgcaaagctgggtacacaggaacacagtgtgaaaggtgtgcaccgggatatttcgggaatccccagaaattcggaggtagctgccaaccatgcagttgtaacagcaatggccagctgggcagctgtcatcccctgactggagactgcataaaccaagaacccaaagatagcagccctgcagaagaatgtgatgattgcgacagctgtgtgatgaccctcctgaacgacctggccaccatgggcgagcagctccgcctggtcaagtctcagctgcagggcctgagtgccagcgcagggcttctggagcagatgaggcacatggagacccaggccaaggacctgaggaatcagttgctcaactaccgttctgccatttcaaatcatggatcaaaaatagaaggcctggaaagagaactgactgatttgaatcaagaatttgagactttgcaagaaaaggctcaagtaaattccagaaaagcacaaacattaaacaacaatgttaatcgggcaacacaaagcgcaaaagaactggatgtgaagattaaaaatgtcatccggaatgtgcacattcttttaaagcagatctctgggacagatggagagggaaacaacgtgccttcaggtgacttttccagagagtgggctgaagcccagcgcatgatgagggaactgcggaacaggaactttggaaagcacctcagagaagcagaagctgataaaagggagtcgcagctcttgctgaaccggataaggacctggcagaaaacccaccagggggagaacaatgggcttgctaacagtatccgggattctttaaatgaatacgaagccaaactcagtgaccttcgtgctcggctgcaggaggcagctgcccaagccaagcaggcaaatggcttgaaccaagaaaacgagagagctttgggagccattcagagacaagtgaaagaaataaattccctgcagagtgatttcaccaagtatctaaccactgcagactcatctttgttgcaaaccaacattgcgctgcagctgatggagaaaagccagaaggaatatgaaaaattagctgccagtttaaatgaagcaagacaagaactaagtgacaaagtaagagaactttccagatctgctggcaaaacatcccttgtggaggaggcagaaaagcacgcgcggtccttacaagagctggcaaagcagctggaagagatcaagagaaacgccagcggggatgagctggtgcgctgtgctgtggatgccgccaccgcctacgagaacatcctcaatgccatcaaagcggccgaggacgcagccaacagggctgccagtgcatctgaatctgccctccagacagtgataaaggaagatctgccaagaaaagctaaaaccctgagttccaacagtgataaactgttaaatgaagccaagatgacacaaaagaagctaaagcaagaagtcagtccagctctcaacaacctacagcaaaccctgaatattgtgacagttcagaaagaagtgatagacaccaatctcacaactctccgagatggtcttcatgggatacagagaggtgatattgatgctatgatcagtagtgcaaagagcatggtcagaaaggccaacgacatcacagatgaggttctggatgggctcaaccccatccagacagatgtggaaagaattaaggacacctatgggaggacacagaacgaagacttcaaaaaggctctgactgatgcagataactcggtgaataagttaaccaacaaactacctgatctttggcgcaagattgaaagtatcaaccaacagctgttgcccttgggaaacatctctgacaacatggacagaatacgagaactaattcagcaggccagagatgctgccagtaaggttgctgtccccatgaggttcaatggtaaatctggagtcgaagtccgactgccaaatgacctggaagatttgaaaggatatacatctctgtccttgtttctccaaaggcccaactcaagagaaaatgggggtactgagaatatgtttgtgatgtaccttggaaataaagatgcctcccgggactacatcggcatggcagttgtggatggccagctcacctgtgtctacaacctgggggaccgtgaggctgaactccaagtggaccagatcttgaccaagagtgagactaaggaggcagttatggatcgggtgaaatttcagagaatttatcagtttgcaaggcttaattacaccaaaggagccacatccagtaaaccagaaacacccggagtctatgacatggatggtagaaatagcaatacactccttaatttggatcctgaaaatgttgtattttatgttggaggttacccacctgattttaaacttcccagtcgactaagtttccctccatacaaaggttgtattgaattagatgacctcaatgaaaatgttctgagcttgtacaacttcaaaaaaacattcaatctcaacacaactgaagtggagccttgtagaaggaggaaggaagagtcagacaaaaattattttgaaggtacgggctatgctcgagttccaactcaaccacatgctcccatcccaacctttggacagacaattcagaccaccgtggatagaggcttgctgttctttgcagaaaacggggatcgcttcatatctctaaatatagaagatggcaagctcatggtgagatacaaactgaattcagagctaccaaaagagagaggagttggagacgccataaacaacggcagagaccattcgattcagatcaaaattggaaaactccaaaagcgtatgtggataaatgtggacgttcaaaacactataattgatggtgaagtatttgatttcagcacatattatctgggaggaattccaattgcaatcagggaaagatttaacatttctacgcctgctttccgaggctgcatgaaaaatttgaagaaaaccagtggtgtcgttagattgaatgatactgtgggagtaaccaaaaagtgctcggaagactggaagcttgtgcgatctgcctcattctccagaggaggacaattgagtttcactgatttgggcttaccacctactgaccacctccaggcctcatttggatttcagacctttcaacccagtggcatattattagatcatcagacatggacaaggaacctgcaggtcactctggaagatggttacattgaattgagcaccagcgatagcggcggcccaatttttaaatctccacagacgtatatggatggtttactgcattatgtatctgtaataagcgacaactctggactacggcttctcatcgatgaccagcttctgagaaatagcaaaaggctaaaacacatttcaagttcccggcagtctctgcgtctgggcgggagcaattttgagggttgtattagcaatgtttttgtccagaggttatcactgagtcctgaagtcctagatttgaccagtaactctctcaagagagatgtgtccctgggaggctgcagtttaaacaaaccaccttttctaatgttgcttaaaggttctaccaggtttaacaagaccaagacttttcgtatcaaccagctgttgcaggacacaccagtggcctccccaaggagcgtgaaggtgtggcaagatgcttgctcaccacttcccaagacccaggccaatcatggagccctccagtttggggacattcccaccagccacttgctattcaagcttcctcaggagctgctgaaacccaggtcacagtttgctgtggacatgcagacaacatcctccagaggactggtgtttcacacgggcactaagaactcctttatggctctttatctttcaaaaggacgtctggtctttgcactggggacagatgggaaaaaattgaggatcaaaagcaaggagaaatgcaatgatgggaaatggcacacggtggtgtttggccatgatggggaaaaggggcgcttggttgtggatggactgagggcccgggagggaagtttgcctggaaactccaccatcagcatcagagcgccagtttacctgggatcacctccatcagggaaaccaaagagcctccccacaaacagctttgtgggatgcctgaagaactttcagctggattcaaaacccttgtataccccttcttcaagcttcggggtgtcttcctgcttgggtggtcctttggagaaaggcatttatttctctgaagaaggaggtcatgtcgtcttggctcactctgtattgttggggccagaatttaagcttgttttcagcatccgcccaagaagtctcactgggatcctaatacacatcggaagtcagcccgggaagcacttatgtgtttacctggaggcaggaaaggtcacggcctctatggacagtggggcaggtgggacctcaacgtcggtcacaccaaagcagtctctgtgtgatggacagtggcactcggtggcagtcaccataaaacaacacatcctgcacctggaactggacacagacagtagctacacagctggacagatccccttcccacctgccagcactcaagagccactacaccttggaggtgctccagccaatttgacgacactgaggatccctgtgtggaaatcattctttggctgtctgaggaatattcatgtcaatcacatccctgtccctgtcactgaagccttggaagtccaggggcctgtcagtctgaatggttgtcctgaccagtaacccaagcctatttcacagcaaggaaattcaccttcaaaagcactgattacccaatgcacctccctccccagctcgagatcattcttcaattaggacacaaaccagacaggtttaatagcgaatctaattttgaattctgaccatggatacccatcactttggcattcagtgctacatgtgtattttatataaaaatcccatttcttgaagataaaaaaattgttattcaaattgttatgcacagaatgtttttggtaatattaatttccactaaaaaattaaatgtctttt

SEQ ID NO: 2 - protein, 1713 aa, Human
MGWLWIFGAALGQCLGYSSQQQRVPFLQPPGQSQLQASYVEFRPSQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHILLKQISGTDGEGNNVPSGDFSREWAEAQRMMRELRNRNFGKHLREAEADKRESQLLLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 3 - DNA, 3930 bp, Human
  CDS (121)...(3630)
  ref <301>: AUTHORS: Gerecke,D.R., Wagman,D.W., Champliaud,M.F. and Burgeson,R.E.
  ref <302>: TITLE: The complete primary structure for a novel laminin chain, the laminin B1k chain
  ref <303>: JOURNAL: J. Biol. Chem.
  ref <304>: VOLUME: 269
  ref <305>: ISSUE: 15
  ref <306>: PAGES: 11073-11080
  ref <307>: DATE: 1994
gggcgggaggaggactgtatctctggatgcctggggcctggtttcagggcctgatttattcctcttcctgggagctcactcaggaaaggtcctttctggggatcaccccattggctgaagatgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcccagaatgatgtgaaccctgtctctctgcagctggacctggacaggagattccagcttcaagaagtcatgatggagttccgagggcccatgcctgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggctccaccgctgtgcaggtccacgatgtctgcgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtcgcaggggctccctgtgacccagtgaccgggcagtgtgtgtgcaaggagcatgtgcagggagagcgctgtgacctatgcaagccgggcttcactggactcacctacgccaacccgcgacggtgccaccgctgtgactgcaacatcctggggtcccgggagatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactccctcagcccacagtgcaaccagttcacagggcagtgcccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgcaatcgctacccggtgtgcgtggcctgccacccttgcttccagacctatgatgcggacctccgggagcaggccctgcgctttggtagactcccgaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggttggagatgtcttcgttgcctgacctgacacccaccttcaacaagctctgtggcaactccaggcagatggcttgcaccccaatatcatgccctggtgagctatgtccccaagacaatggcacagcctgtgcgtcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaggatgtggttgggaacctgcggcaggggacagtggcactgcaggaagctcaggacaccatgcaaggcaccagccggtcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtgatgctacacgttccagcccgttgccccactcatctgcgcgctttgcttttggttggggggcagattgggttggaatgctttccatctccaggagactttcatgtagcccaaagtacagcctggaccacccctggtgtgtgtagctagtaagattaccctgagctgcagctgagcctgagccaatgggacagttacacttgacagacaaagatggtggagattggcatgccattgaaactaagagctctcaagtcaaggaagctgggctgggcagtatcccccgcctttagttctccac

SEQ ID NO: 4 - protein, 1170 aa, Human [sequence omitted]

SEQ ID NO: 5 - DNA, 5200 bp, Human
  CDS (118)...(3696)
  ref <301>: AUTHORS: Kallunki,P., Sainio,K., Eddy,R., Byers,M., Kallunki,T., Sariola,H., Beck,K., Hirvonen,H., Shows,T.B. and Tryggvason,K.
  ref <302>: TITLE: A truncated laminin chain homologous to the B2 chain: structure, spatial expression, and chromosomal assignment
  ref <303>: JOURNAL: J. Cell Biol.
  ref <304>: VOLUME: 119
  ref <306>: PAGES: 679-693
  ref <307>: DATE: 1992
gaccacctgatcgaaggaaaaggaaggcacagcggagcgcagagtgagaaccaccaaccgaggcgccgggcagcgacccctgcagcggagacagagactgagcggcccggcaccgccatgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactctggacggtgcagctgtaaaccaggtgtgacaggagccagatgcgaccgatgtctgccaggcttccacatgctcacggatgcggggtgcacccaagaccagagactgctagactccaagtgtgactgtgacccagctggcatcgcagggccctgtgacgcgggccgctgtgtctgcaagccagctgttactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagatcctgtctattttgtggctcctgccaaatttcttgggaatcaacaggtgagctatgggcaaagcctgtcctttgactaccgtgtggacagaggaggcagacacccatctgcccatgatgtgatcctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgattccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacagcaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctccgcttcagggagtcagtgatcagtcctttcaggtggaagaagcaaagaggatcaaacaaaaagcggattcactctcaagcctggtaaccaggcatatggatgagttcaagcgtacacaaaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatggaccagcctctcagtgtagatgaagaggggctggtcttactggagcagaagctttcccgagccaagacccagatcaacagccaactgcggcccatgatgtcagagctggaagagagggcacgtcagcagaggggccacctccatttgctggagacaagcatagatgggattctggctgatgtgaagaacttggagaacattagggacaacctgcccccaggctgctacaatacccaggctcttgagcaacagtgaagctgccataaatatttctcaactgaggttcttgggatacagatctcagggctcgggagccatgtcatgtgagtgggtgggatggggacatttgaacatgtttaatgggtatgctcaggtcaactgacctgaccccattcctgatcccatggccaggtggttgtcttattgcaccatactccttgcttcctgatgctgggcatgaggcagataggcactggtgtgagaatgatcaaggatctggaccccaaagatagactggatggaaagacaaactgcacaggcagatgtttgcctcataatagtcgtaagtggagtcctggaatttggacaagtgctgttgggatatagtcaacttattctttgagtaatgtgactaaaggaaaaaactttgactttgcccaggcatgaaattcttcctaatgtcagaacagagtgcaacccagtcacactgtggccagtaaaatactattgcctcatattgtcctctgcaagcttcttgctgatcagagttcctcctacttacaacccagggtgtgaacatgttctccattttcaagctggaagaagtgagcagtgttggagtgaggacctgtaaggcaggcccattcagagctatggtgcttgctggtgcctgccaccttcaagttctggacctgggcatgacatcctttcttttaatgatgccatggcaacttagagattgcatttttattaaagcatttcctaccagcaaagcaaatgttgggaaagtatttactttttcggtttcaaagtgatagaaaagtgtggcttgggcattgaaagaggtaaaattctctagatttattagtcctaattcaatcctacttttcgaacaccaaaaatgatgcgcatcaatgtattttatcttattttctcaatctcctctctctttcctccacccataataagagaatgttcctactcacacttcagctgggtcacatccatccctccattcatccttccatccatctttccatccattacctccatccatccttccaacatatatttattgagtacctactgtgtgccaggggctggtgggacagtggtgacatagtctctgccctcatagagttgattgtctagtgaggaagacaagcatttttaaaaaataaatttaaacttacaaactttgtttgtcacaagtggtgtttattgcaataaccgcttggtttgcaacctctttgctcaacagaacatatgttgcaagaccctcccatgggcactgagtttggcaaggatgacagagctctgggttgtgcacatttctttgcattccagcgtcactctgtgccttctacaactgattgcaacagactgttgagttatgataacaccagtgggaattgctggaggaaccagaggcacttccaccttggctgggaagactatggtgctgccttgcttctgtatttccttggattttcctgaaagtgtttttaaataaagaacaattgttagatgccaaaaa

SEQ ID NO: 6 - protein, 1193 aa, Human [sequence omitted]

SEQ ID NO: 7 - DNA, 20 bp, Artificial Sequence
  primer for PCR amplification
aagcagaagatgcggactgt

SEQ ID NO: 8 - DNA, 20 bp, Artificial Sequence
  primer for PCR amplification
accactggtttttctgccac

SEQ ID NO: 9 - DNA, 23 bp, Artificial Sequence
  primer for PCR amplification
tctttccaccaggcccccggctc

SEQ ID NO: 10 - DNA, 23 bp, Artificial Sequence
  primer for PCR amplification
tgcgggcggacatggggagatcc

SEQ ID NO: 11 - DNA, 23 bp, Artificial Sequence
  primer for PCR amplification
tagagctagactccgggcgatga

SEQ ID NO: 12 - DNA, 23 bp, Artificial Sequence
  primer for PCR amplification
ttgccttaaacaagaccacgaaa

SEQ ID NO: 13 - DNA, 20 bp, Artificial Sequence
  primer for PCR amplification
caccatggagaaggccgggg

SEQ ID NO: 14 - DNA, 20 bp, Artificial Sequence
  primer for PCR amplification
gacggacacattgggggtag

(19) United States
(12) Patent Application Publication
(10) Pub. No.: US 2013/0164839 A1
(43) Pub. Date: Jun. 27, 2013

(54) METHOD FOR CULTURING CELLS IN A SYSTEM COMPRISING LAMININ-5

(71) Applicants: Hisataka Yasuda, Tokyo (JP); Munehiro Yamada, Nagahama-shi (JP); Yukiko Taketani, Nagahama-shi (JP); Yoshiya Tomimori, Nagahama-shi (JP); Kaoru Mori, Nagahama-shi (JP)

(72) Inventors: Hisataka Yasuda, Tokyo (JP); Munehiro Yamada, Nagahama-shi (JP); Yukiko Taketani, Nagahama-shi (JP); Yoshiya Tomimori, Nagahama-shi (JP); Kaoru Mori, Nagahama-shi (JP)

(73) Assignee: ORIENTAL YEAST CO., LTD., Tokyo (JP)

(21) Appl. No.: 13/638,172

(22) PCT Filed: Mar. 31, 2011
(86) PCT No.: PCT/JP2011/058265

§ 371 Date: Sep. 28, 2012

(30) Foreign Application Priority Data
Mar. 31, 2010 (JP) 2010-084153

(51) Int. Cl. C12N 5/071; C12N 5/0735; C12N 5/0775; C12N 5/0786; C12N 5/0797; C12N 5/077; C12N 5/078; C12N 5/09; C12N 5/0789

(52) U.S. Cl. 435/353; 435/375; 435/377; 435/354; 435/366; 435/406

(57) ABSTRACT

The present invention is directed to providing a method for culturing cells in a system containing laminin-5. The method of the present invention is characterized by a culture system containing a polypeptide selected from a group consisting of: a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone.

### TECHNICAL FIELD

The present invention relates to a method for culturing cells in a system containing laminin-5.

### BACKGROUND ART

Laminin, which is localized primarily on the basement membranes of various tissues, is an extracellular matrix protein playing an important role maintaining tissue structure and in controlling cell functions (Matrix Biol., 18:19-28, 1999, Dev. Dyn., 218:213-234, 2000).

Laminin is structured as a heterotrimer molecule composed of α, β and γ chains linked to each other via disulfide linkages, which takes a characteristic cross-structure. Each chain is composed of multiple domains, and domains I and II form a triple helix. Before the filing of the present application, at least 15 isoforms of laminin molecules have been identified from different combinations of 5 types of α chains (α1 to α5), 3 types of βchains (β1 to β3) and 3 types of γ chains (γ1 to γ3), and it is suggested that there are actually several times that number of isoforms (Cancer Sci., 97:91-98, 2006; Dev. Dyn., 218:213-234, 2000; J. Neurosci., 20:6517-6528, 2000; Physiol Rev. 85:979-1000, 2005). These α, β and γ chains are encoded by different genes, respectively; and the individual laminin isoforms have specific sites of localization and specific functions, and mainly regulate cell adhesion, proliferation, motility, differentiation and so on through the cell membrane receptor integrin (Dev. Dyn. 218:213-234, 2000, Physiol. Rev. 85:979-1000, 2005).

For example, concerning localization in vivo, α2 chains center around the muscle or the nervous tissue, whereas α3 chains center around the skin tissue. The chains differ in their functions as well; specifically, a gene abnormality in the α2 chain causes muscular dystrophy, whereas the gene abnormality in the α3 chain causes a serious symptom known as junctional epidermolysis bullosa (Dev. Dyn. 218:213-234, 2000). In addition, the chains exhibit completely different functions from each other in an in vitro experiment. Laminin 2 and laminin 4, which comprise an α2 chain as their component chain, exhibit almost no adhesion activity against mesenchymal stem cells, whereas laminin-5, which comprise an α3 chain as its component chain, exhibits extremely strong adhesion activity to such cells (Stem Cell. 24:2346-2354, 2006). Differences in the component α, β, γ chains lead to differences in the functions and activities of the laminin isoforms.

Fifteen laminin molecular species and the corresponding subunit arrangement of each species are shown in Table 1.

TABLE 1
Laminin molecular species and subunit structure

| Name | Structure | Also called |
|---|---|---|
| Laminin-1 | α1β1γ1 | EHS laminin |
| Laminin-2 | α1β1γ1 | Merosin |
| Laminin-3 | α1β2γ1 | S-Laminin |
| Laminin-4 | α2β2γ1 | S-Merosin |
| Laminin-5 | α3β3γ2 | Ladsin/epiligrin/ |
| | | kalinin/nicein |
| Laminin-6 | α3β1γ1 | K-Laminin |
| Laminin-7 | α3β2γ1 | KS-Laminin |
| Laminin-8 | α4β1γ1 | |
| Laminin-9 | α4β2γ1 | |
| Laminin-10 | α5β1γ1 | |
| Laminin-11 | α5β2γ1 | |
| Laminin-12 | α2β1γ3 | |
| Laminin-13 | α3β2γ3 | |
| Laminin-14 | α4β2γ3 | |
| Laminin-15 | α5β2γ3 | |

Laminin molecules form a basement membrane through associating with each other or to other matrix molecules by the amino (N) terminal sections (short arms) of the three chains. Meanwhile, the carboxy (C) terminal of the α chain comprises five homologous spherical domains (G1-G5 domains or LG1-LG5), as primary sites for bonding with integrin and other receptors.

Laminin-5

Laminin-5 (also known as kalinin, epiligrin, nicein, ladsin), which is a laminin isoform composed of an α3 chain, a β3 chain and a γ2 chain, was discovered by different research institutes through different routes. (J. Cell Biol. 114:567-576, 1991; Cell 65:599-610, 1991; J. Invest Dermatol. 101:738-743, 1993; Proc. Natl. Acad. Sci. USA 90:11767-11771, 1993).

Laminin-5 is reported to have strong cell adhesion activity, cell scattering activity, cell proliferation activity and the like against various cells (Proc. Natl. Acad. Sci. USA. 90:11767-11771, 1993; J. Biochem. 116:862-869, 1994; J. Cell Biol. 125:205-214, 1994; Mol. Biol. Cell. 16:881-890, 2005, Stem Cell. 24:2346-2354, 2006). WO 2007/023875 discloses culture techniques using laminin-5 for mesenchymal stem cells.

However, there was no general efficient method for enhancing various activities of laminin-5 before the present invention.

### CITATION LIST

### Patent Documents

[Patent Document 1] International Publication WO 2007/023875
[Patent Document 2] International Publication WO 2009/123349

### Non-Patent Documents

[Non-Patent Document 1] Matrix Biol., 18:19-28, 1999
[Non-Patent Document 2] Dev. Dyn., 218:213-234, 2000
[Non-Patent Document 3] Cancer Sci., 97:91-98, 2006
[Non-Patent Document 4] J. Neurosci., 20:6517-6528, 2000
[Non-Patent Document 5] Physiol. Rev. 85:979-1000, 2005
[Non-Patent Document 6] J. Cell Biol. 114:567-576, 1991
[Non-Patent Document 7] Cell. 65:599-610, 1991
[Non-Patent Document 8] J. Invest Dermatol. 101:738-743, 1993
[Non-Patent Document 9] Proc. Natl. Acad. Sci. USA. 90:11767-11771, 1993
[Non-Patent Document 10] J. Biochem. 116:862-869, 1994
[Non-Patent Document 11] J. Cell Biol. 125:205-214, 1994
[Non-Patent Document 12] Mol. Biol. Cell. 16:881-890, 2005
[Non-Patent Document 13] Stem Cell. 24:2346-2354, 2006
[Non-Patent Document 14] Dev. Biol. 163: p. 288-292, 1994
[Non-Patent Document 15] J. Cell Sci. 112:1-10, 1999
[Non-Patent Document 16] Exp. Cell Res. 310:256-269, 2005
[Non-Patent Document 17] J. Cell Sci. 119:3206-3218, 2006

### SUMMARY OF INVENTION

### Technical Problem

The object of the present invention is to provide a technology for increasing the activity of laminin-5 in a method for culturing cells in a system containing laminin-5.

The present inventors found that various activities of laminin-5 increase by the combined use of specific polypeptides with laminin-5 in a method for culturing cells in a system containing laminin-5, and conceived the present invention.

### Solution to Problem

The present invention comprises the following preferable embodiments.

### Embodiment 1

A method for culturing cells in a system containing laminin-5 characterized by a culture system comprising a polypeptide selected from a group consisting of: a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone.

### Embodiment 2

The method according to Embodiment 1, wherein the protein belonging to the tumor necrosis factor (TNF) family is a receptor activator NFₖ B ligand (RANKL).

### Embodiment 3

The method according to Embodiment 1, wherein peptone is selected from a group consisting of a cotton seed derived peptone, a soy bean derived peptone, a wheat derived peptone and a pea derived peptone.

### Embodiment 4

The method according to any one of Embodiments 1 to 3, wherein a cell culture vessel is treated with laminin-5 after it is treated with polypeptide, or the vessel is treated with polypeptide and laminin at the same time.

### Embodiment 5

The method according to any one of Embodiments 1 to 4, wherein an activity of laminin-5 against the cells, selected from a group consisting of a cell adhesion activity, a cell scattering activity, a wound healing activity, a proliferation stimulating activity, an activity for maintaining undifferentiated-state and an activity for maintaining pluripotency is increased.

### Embodiment 6

The method according to any one of Embodiments 1 to 5, wherein the cells are selected from a group consisting of pluripotent stem cells, tissue stem cells, somatic cells, germ cells and sacroma cells.

### Embodiment 7

The method according to Embodiment 6, wherein the pluripotent stem cells are selected from embryonic stem cells, induced pluripotent stem cells, embryonic germ cells or germline stem cells;

the tissue stem cells are selected from mesenchymal stem cells, hepatic stem cells, pancreatic stem cells, neural stem cells, skin stem cells or hematopoietic stem cells; or

the somatic cells are selected from hepatic cells, pancreatic cells, muscle cells, osteocytes, osteoblasts, osteoclasts, cartilage cells, fat cells, skin cells, fibroblasts, pancreatic cells, kidney cells, pneumocytes or blood cells, which are lymphocites, red blood cells, white blood cells, monocytes, macrophage or megakaryocytes.

### Embodiment 8

The method according to any one of Embodiments 1 to 5, wherein the cells are derived from a species selected from a group consisting of a mouse, a rat or a human.

### Embodiment 9

The method according to any one of Embodiments 1 to 8, wherein the polypeptide is used at a concentration of 1 μg/ml to 200 μg/ml.

### Embodiment 10

The method according to any one of Embodiments 1 to 8, wherein the polypeptide is used at a concentration of 3.125 μg/ml to 12.5 μg/ml.

### Embodiment 11

The method according to any one of Embodiments 1 to 8, wherein the cell culture system comprises two or more types of polypeptides.

### Embodiment 12

A composition to be used in a method for culturing cells in a system containing laminin-5, wherein the composition comprises a polypeptide selected from a group consisting of: a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone.

### Embodiment 13

The composition according to Embodiment 12, which further comprises laminin-5.

### Embodiment 14

A kit for use in a method for culturing cells in a system containing laminin-5, wherein the system contains a polypeptide selected from a group consisting of: a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone.

### Embodiment 15

The kit according to Embodiment 14 which further comprises laminin-5.

### Advantageous Effect of Invention

In the present invention, a combined use of laminin-5 and specific polypeptides increases the activity of laminin-5 against a cell, which is selected from a group consisting of a cell adhesion activity, a cell scattering activity, a wound healing activity, a proliferation stimulating activity, an activity for maintaining undifferentiated-state and an activity for pluripotency.

### BRIEF DESCRIPTION OF DRAWINGS

FIG. 1 shows electrophoresis of purified recombinant human laminin-5 on an SDS polyacrylamide gel. It should be noted that the right lane in FIG. 1 shows the results of electrophoresis of 1 μg of recombinant human laminin-5.

FIG. 2 shows the effect of recombinant human laminin-5 (0.25 μg/ml) and various proteins in blood on the cell adhesion activity against BRL cells. FIGS. 2A-D show results for each of human serum albumin (HSA), bovine serum albumin (BSA), human serum (HS) and IgG, at the concentrations shown in the figures (0-800 μg/ml).

FIG. 3 shows the result of an investigation for the optimal concentration of HSA to increase the cell adhesion activity of the recombinant human laminin-5 at concentrations provided on the x-axis (0-2 μg/ml) against BRL cells.

FIG. 3A

Black diamond: just recombinant human laminin-5

White square: recombinant human laminin-5+HSA 0.78125 μg/ml

White triangle: recombinant human laminin-5+HSA 3.125 μg/ml

Cross: recombinant human laminin-5+HSA 12.5 μg/ml

FIG. 3B

Black diamond: just recombinant human laminin-5

White square: recombinant human laminin-5+HSA 12.5 μg/ml

White triangle: recombinant human laminin-5+HSA 50 μg/ml

Cross: recombinant human laminin-5+HSA 200 μg/ml

FIG. 4 shows the result of an investigation for the optimal concentration of recombinant HSA (rHSA) to increase the cell adhesion activity of the recombinant human laminin-5 at concentrations provided on the x-axis (0-2 μg/ml) against BRL cells.

FIG. 4A

Black diamond: just recombinant human laminin-5

White square: recombinant human laminin-5+rHSA 0.78125 μg/ml

White triangle: recombinant human laminin-5+rHSA 3.125 μg/ml

Cross: recombinant human laminin-5+rHSA 12.5 g/ml

FIG. 4B

Black diamond: just recombinant human laminin-5

White square: recombinant human laminin-5+rHSA 12.5 g/ml

White triangle: recombinant human laminin-5+rHSA 50 μg/ml

Cross: recombinant human laminin-5+rHSA 200 μg/ml

FIG. 5 shows the result of an investigation on the elevating effects of gelatin (Gel), sRANKL and peptine (Pep) on the cell adhesion activity of the recombinant human laminin-5 at concentrations provided on the x-axis (0-2 μg/ml) against BRL cells.

FIG. 5A

Black diamond: just recombinant human laminin-5

White square: recombinant human laminin-5+HSA 10 μg/ml

White triangle: recombinant human laminin-5+Gel 10 μg/ml

FIG. 5B

Black diamond: just recombinant human laminin-5

White square: recombinant human laminin-5+HSA 12.5 μg/ml

White triangle: recombinant human laminin-5+sRANKL 50 μg/ml

FIG. 5C

Black diamond: just recombinant human laminin-5

White square: recombinant human laminin-5+HSA 10 μg/ml

White triangle: recombinant human laminin-5+Pep 10 μg/ml

FIG. 6 shows the result of an investigation on the elevating effects of Pep on the cell adhesion activity of the recombinant human laminin-5 at concentrations provided on the x-axis (0-2 μg/ml) against BRL cells.

Black diamond: just recombinant human laminin-5

White diamond: recombinant human laminin-5+Pep 15.6 μg/ml

Black square: recombinant human laminin-5+Pep 62.5 μg/ml

White square: recombinant human laminin-5+Pep 250 μg/ml

White triangle: recombinant human laminin-5+Pep 1000 μg/ml

FIG. 7 shows the result of an investigation on whether recombinant human laminin-5 (0.25 μg/ml) and various sugars have effects on the cell adhesion activity against BRL cells. FIGS. 7A-D respectively show the results, each for xylose (Xyl), trehalose (Tre), mannose (Man) and lactose (Lac) at the concentrations shown in the figures (0-10 μg/ml).

FIG. 8 shows the result of an investigation on whether recombinant human laminin-5 (0.25 g/ml) and various sugars affect the cell adhesion activity against BRL cells at a higher concentration. FIG. 8A shows results for Tre and Man each at 100 μg/ml.

FIG. 8B shows results for Xyl and Lac at 100 μg/ml.

FIG. 9 shows the result of an investigation on whether recombinant human laminin-5 (0.25 μg/ml) and various amino acids are effective on the cell adhesion activity against BRL cells.

FIG. 9A

Black diamond: just recombinant human laminin-5 (control)

White square: recombinant human laminin-5+HSA 10 μg/ml

White triangle: recombinant human laminin-5+glycine (Gly) 10 μg/ml

Grey triangle: recombinant human laminin-5+Gly 100 μg/ml

Black triangle: recombinant human laminin-5+Gly 1000 μg/ml

FIG. 9B

Black diamond: just recombinant human laminin-5 (control)

White square: recombinant human laminin-5+HSA 10 μg/ml

White triangle: recombinant human laminin-5+arginine (Arg) 10 μg/ml

Grey triangle: recombinant human laminin-5+Arg 100 μg/ml

Black triangle: recombinant human laminin-5+Arg 1000 μg/ml

FIG. 10 shows a synergetic effect of recombinant human laminin-5 on the cell adhesion activity when two types of proteins in blood are used. The proteins in blood on the x-axis were each used at the indicated concentration.

FIG. 11 shows the result of an assessment to determine the order of treating the cell incubator. Recombinant human laminin-5 of concentrations provided on the x-axis (0-2 μg/ml) was used.

Black diamond: just rLm5

White square: the cell incubation plate was treated with rLm5 after it was treated with rHSA (rHSA→rLm5)

White triangle: the cell incubation plate was treated with rHSA and rLm5 at the same time (rLm5+rHSA)

Cross: the cell incubation plate was treated with rHSA after it was treated with rLm5 (rLm5→rHSA)

FIG. 12 shows the result of an investigation for the optimal concentration of recombinant HSA (rHSA) to increase the cell adhesion activity of the recombinant human laminin-5 at concentrations provided on the x-axis (0-2 μg/ml) against HT1080 cells.

FIG. 12A

Black diamond: just recombinant human laminin-5 (control)

White square: recombinant human laminin-5+rHSA 0.78125 μg/ml

White triangle: recombinant human laminin-5+rHSA 3.125 μg/ml

Cross: recombinant human laminin-5+rHSA 12.5 μg/ml

FIG. 12B

Black diamond: just recombinant human laminin-5 (control)

White square: recombinant human laminin-5+rHSA 12.5 μg/ml

White triangle: recombinant human laminin-5+rHSA 50 μg/ml

Cross: recombinant human laminin-5+rHSA 200 μg/ml

FIG. 13 shows the result of an investigation for the optimal concentration of recombinant HSA (rHSA) to increase the cell adhesion activity of the recombinant human laminin-5 at concentrations provided on the x-axis (0-2 μg/ml) against human mesenchymal stem cells (hMSC).

FIG. 13A

Black diamond: just recombinant human laminin-5 (control)

White square: recombinant human laminin-5+rHSA 0.78125 μg/ml

White triangle: recombinant human laminin-5+rHSA 3.125 μg/ml

Cross: recombinant human laminin-5+rHSA 12.5 μg/ml

FIG. 13B

Black diamond: just recombinant human laminin-5 (control)

White square: recombinant human laminin-5+rHSA 12.5 g/ml

White triangle: recombinant human laminin-5+rHSA 50 μg/ml

Cross: recombinant human laminin-5+rHSA 200 μg/ml

FIG. 14 shows the result of an investigation for the optimal concentration of recombinant HSA (rHSA) to increase the cell adhesion activity of the recombinant human laminin-5 at concentrations provided on the x-axis (0-16 μg/ml) against EB3 cells.

FIG. 14A

Black diamond: just recombinant human laminin-5 (control)

White square: recombinant human laminin-5+rHSA 0.78125 μg/ml

White triangle: recombinant human laminin-5+rHSA 3.125 μg/ml

Cross: recombinant human laminin-5+rHSA 12.5 μg/ml

FIG. 14B

Black diamond: just recombinant human laminin-5 (control)

White square: recombinant human laminin-5+rHSA 12.5 μg/ml

White triangle: recombinant human laminin-5+rHSA 50 μg/ml

Cross: recombinant human laminin-5+rHSA 200 μg/ml

FIG. 15 consists of photographs showing the cell state of recombinant human laminin-5, at concentrations provided in the figure, after analyzing the adhesion of EB3 cells for cases with rHSA (12.5 μg/ml) added to it. The photographs on the left are results of not using rHSA (control), and those on the right are results of using rHSA.

FIG. 16 shows the result of an investigation on whether HSA exhibits elevating effects on the cell adhesion activity against laminin isoforms other than human laminin-5, as it does for laminin-5. Laminin-5 was used at concentrations provided on the x-axis (0-2 μg/ml).

Black diamond: just recombinant human laminin-5 (control)

White diamond: recombinant human laminin-5+HSA 10 μg/ml

FIG. 17 shows the result of an investigation on whether rHSA exhibits elevating effects on the cell adhesion activity against extracellular matrix proteins and isoforms other than human laminin-5, as it does for laminin-5. Vitronectin (Vn/SIGMA) was used as the other extracellular matrix protein, and laminin 2 (Lm2/Millipore) was used as the other laminin isoform. Laminin-5 (0-2 μg/ml) and vitronection (0-32 μg/ml), Lm2 (0-32 μg/ml) were each used at concentrations provided on the x-axis.

FIG. 17A

Black diamond: just recombinant human laminin-5 (control)

White diamond: recombinant human laminin-5+rHSA 10 μg/ml

Black circle: just Vn (control)

White circle: Vn+rHSA 10 μg/ml

FIG. 17B

Black diamond: just recombinant human laminin-5 (control)

White diamond: recombinant human laminin-5+rHSA 10 μg/ml

Black square: just Lm2 (control)

White square: Lm2+rHSA 10 μg/ml

FIG. 18 shows a result of an investigation on the activity elevating effects of rHSA (10 μg/ml) on the cell scattering activity of recombinant human laminin-5 at concentrations provided on the x-axis against BRL cells. The activity elevating effects of rHSA was observed to be especially high when the concentration of rLm5 was at 0.02 μg/ml.

FIG. 19 is photographs showing the result of an investigation on the activity elevating effects of rHSA (10 μg/ml) concerning the cell scattering activity of recombinant human laminin-5 at the given concentrations (0-0.2 μg/ml) against BRL cells.

FIG. 20 shows the result of an investigation on the activity elevating effects of rHSA (10 μg/ml) concerning the wound healing activity of recombinant human laminin-5 at concentrations provided on the x-axis (0-0.1 g/ml) against BRL cells.

FIG. 21 is photographs showing the result of an investigation on the activity elevating effects of rHSA (10 μg/ml) concerning the wound healing activity of recombinant human laminin-5 at concentrations (0-0.1 g/ml) against BRL cells.

FIG. 22 shows the result of an investigation on the activity elevating effects of rHSA (10 μg/ml) concerning the proliferation activity of recombinant human laminin-5 against hMBC cells.

Cross: only serums without coating

White square: Panexin is added (P)

Black triangle: P/rLm5 1 μg/ml

White triangle: P+F (bFGF)/no coating

White circle: P+F/rLm5 1 μg/ml

White diamond: P+F/rLm5 0.2 μg/ml

Black square: P+F/rLm5 0.2 μg/ml+rHSA 10 μg/ml

FIG. 23 shows the result of an investigation on the activity elevating effects of rHSA (10 μg/ml) concerning the proliferation activity of recombinant human laminin-5 against EB3 cells.

White diamond: Lm5 (2 μg/ml)

Black square: Lm5 (0.2 μg/ml)+rHSA 12.5 μg/ml

Grey square: Lm5 (0.2 μg/ml)+rHSA 3.125 g/ml

White square: Lm5 (0.2 μg/ml)

FIG. 24 shows the result of an investigation on the activity elevating effects of rHSA concerning the proliferation activity of recombinant human laminin-5 against EB3 cells for various cell supporting materials that are used.

Black circle: proliferation medium (S)+bovine gelatin (G)

Black square: KSR-GMEM (K)+Lm5 (L) (0.05 μg/ml)+rHSA (H) (12.5 μg/ml)

White square: KSR-GMEM (K)+Lm5 (L) (0.05 μg/ml)

FIG. 25 shows the result of an undifferentiated-state marker detection concerning S+G and K+L (0.05 μg/ml)+H (12.5 μg/ml), which both exhibited proliferation in FIG. 24.

### DESCRIPTION OF EMBODIMENTS

The present invention relates to a method for culturing cells in a system containing laminin-5.

The method of the present invention is a method for culturing cells in a system containing laminin-5, characterized by a culture system comprising a polypeptide selected from a group consisting of: a protein in blood other than extracellular matrix proteins, which is, serum, serum albumin, prealbumin, immunoglobulin, α-globulin, 3-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone.

Laminin-5

The method of the present invention is directed to the culture of pluripotent stem cells and its most remarkable feature lies in culturing the pluripotent stem cells in a system containing laminin-5.

The adhesion activity of Laminin-5 against many cell types is reported to be stronger than those of various extracellular matrix proteins including other laminin isoforms (J. Biochem. 116:862-869, 1994; J. Cell Biol. 125:205-214, 1994; Mol. BiolCell. 16:881-890, 2005).

As shown in Table 1, laminin-5 is a laminin molecule composed of α3, β3 and γ2 chains, which plays a dominant role in binding epidermis and corium, and binds preferentially to integrin α3β1 in most cells and also binds to integrin α6β1 or α6β4 in some cells. In laminin-5, it has been elucidated that the α3G2A sequence (RERFNISTPAFRGCMKNLKKTS) in the α3 chain G2 domain and the KRD sequence in the G3 domain are the major binding sites for integrin.

It is also known that laminin-5, after being secreted as a trimer, receives limited proteolysis by protease to remove G4 and G5 domains located at the C-terminal of the α3 chain, and is thereby converted from 190 kDa (nontruncated) into 160 kDa (truncated). Laminin-5 isolated in a standard manner does not have G4 and G5 domains. Such α3 chain-truncated laminin-5 is known to have higher stimulating activities on cell adhesion, motility and neuranagenesis, when compared to non-truncated laminin-5 (J. Biol. Chem., 280 (2005): 14370-14377).

Laminin-5 in the present invention is not particularly limited, and may be either in a non-truncated form containing G4 and G5 domains or in a truncated form free from all or part of G4 and G5 domains.

Moreover, the laminin-5 protein may be either naturally occurring or modified to have one or more modified amino acid residues while maintaining its biological activities, particularly stimulating activity on cell adhesion. Moreover, the laminin-5 protein in the present invention may be of any origin and may be prepared in any manner, as long as it has the features described herein. Namely, the laminin-5 protein of the present invention may be naturally occurring, expressed from recombinant DNA by genetic engineering procedures, or chemically synthesized.

The laminin-5 protein may be of any origin, preferably of human origin. In a case where human pluripotent stem cells are cultured in order to obtain materials for regenerative medicine, etc., it is preferred to use laminin-5 of human origin for the sake of avoiding the use of materials derived from other animals.

SEQ ID NOs: 1 to 6 in the Sequence Listing herein show the nucleotide and amino acid sequences of human laminin-5 α3 chain, the nucleotide and amino acid sequences of human laminin-5 β3 chain and the nucleotide and amino acid sequences of human laminin-5 γ2 chain, respectively. The laminin-5 protein to be used in the present invention is preferably a protein composed of the following subunits: an α3 chain having the amino acid sequence of SEQ ID NO: 2 or an amino acid sequence comprising deletion, addition or substitution of one or more amino acids in the sequence of SEQ ID NO:2 (amino acid residues 1-1713) (J. Biol. Chem. 269:22779-22787, 1994), a β3 chain having the amino acid sequence of SEQ ID NO: 4 or an amino acid sequence comprising deletion, addition or substitution of one or more amino acids in the sequence of SEQ ID NO: 4 (amino acid residues 1-1170) (J. Biol. Chem. 269:11073-11080, 1994), and a γ2 chain having the amino acid sequence of SEQ ID NO: 6 or an amino acid sequence comprising deletion, addition or substitution of one or more amino acids in the sequence of SEQ ID NO: 6 (amino acid residues 1-1193) (J. Cell. Biol. 119:679-693, 1992).

Globular domains (G1 to G5 domains) in the α3 chain correspond to amino acid residues 794-970, 971-1139, 1140-1353, 1354-1529 and 1530-1713, respectively, in SEQ ID NO: 1.

Each chain of laminin-5 may have an amino acid sequence comprising deletion, addition or substitution of one or more amino acid residues in the amino acid sequence shown in the corresponding SEQ ID NO. Such proteins having amino acid sequences homologous to naturally occurring proteins can also be used in the present invention. The number of modifiable amino acids is not particularly limited in the amino acid sequences of α3, β3 and γ2 chains, but it is preferably 1 to 300 amino acid residues, 1 to 200 amino acid residues, 1 to 150 amino acid residues, 1 to 120 amino acid residues, 1 to 100 amino acid residues, 1 to 80 amino acid residues, 1 to 50 amino acid residues, 1 to 30 amino acid residues, 1 to 20 amino acid residues, 1 to 15 amino acid residues, 1 to 10 amino acid residues, or 1 to 5 amino acid residues. More preferably, it is a number of amino acid residues modifiable by known site-directed mutagenesis, for example, 1 to 10 amino acid residues, or 1 to 5 amino acid residues.

It is well known in the art that conservative substitution of amino acids can be used to obtain proteins or polypeptides maintaining their original functions. Such substitution includes replacement of an amino acid with another residue having similar physical and chemical properties, as exemplified by replacement of one fatty acid residue (Ile, Val, Leu or Ala) with another, or replacement between basic residues Lys and Arg, between acidic residues Glu and Asp, between amide residues Gln and Asn, between hydroxyl residues Ser and Tyr, or between aromatic residues Phe and Tyr.

Laminin-5 to be used in the present invention may also be a protein sharing at least 80%, 85%, 90%, 95%, 98% or 99% identity with the amino acid sequences shown in SEQ ID NOs: 2, 4 and 6 and having the ability to stimulate cell adhesion activity.

Identity is calculated as follows: the number of identical residues is divided by the total number of residues in a corresponding known sequence or a domain therein, and then multiplied by 100. Computer programs available for use in the determination of sequence identity using standard parameters include, for example, Gapped BLAST PSI-BLAST (Nucleic Acids Res. 25:3389-3402, 1997), BLAST (J. Mol. Biol. 215:403-410, 1990), and Smith-Waterman (J. Mol. Biol. 147:195-197, 1981). In these programs, default settings are preferably used, but these settings may be modified, if desired.

The laminin-5 protein in the present invention may be of any origin and may be prepared in any manner, as long as it has the features described herein. Namely, the laminin-5 protein of the present invention may be a naturally occurring laminin-5 protein as found in or purified from the supernatant of human or animal cells secreting laminin-5. However, laminin-5 can be effectively produced as a recombinant protein by expressing each subunit using recombinant DNA technology known in the art. It is particularly preferred to obtain laminin-5 as a human recombinant protein, for the sake of avoiding unwanted factors derived from other animals.

For this purpose, primers may be designed based on a DNA sequence comprising nucleic acid residues 1-5139 in SEQ ID NO: 1 (encoding the laminin-5 α3 chain) and nucleotide sequences of nucleic acid residues 121-3630 in SEQ ID NO: 3 (encoding the β3 chain) and nucleic acid residues 118-3696 in SEQ ID NO: 5 (encoding the γ2 chain), and an appropriate cDNA library may be used as a template in polymerase chain reaction (PCR) to amplify desired sequences. Such PCR procedures are well known in the art and can be found, e.g., in “PCR Protocols, A Guide to Methods and Applications,” Academic Press, Michael, et al., 1990.

DNA encoding a gene of each chain of laminin-5 may be integrated into an appropriate vector and then introduced into either eukaryotic or prokaryotic cells by using an expression vector that allows expression in each host, whereby the respective chains are expressed to obtain a desired protein. Host cells which can be used to express laminin-5 are not limited in any way and include prokaryotic host cells such as E. coli and Bacillus subtilis, as well aseukaryotic hosts such as yeast, fungi, insect cells and mammalian cells.

Vector constructed to express laminin-5 can be introduced into the above host cells by transformation, transfection, conjugation, protoplast fusion, electroporation, particle gun technique, calcium phosphate precipitation, direct microinjection or other techniques. The cells containing the vector may be grown in an appropriate medium to produce a laminin-5 protein to be used in the present invention, which may then be purified from the cells or medium to obtain the laminin-5 protein. Purification may be accomplished, for example, by size exclusion chromatography, HPLC, ion exchange chromatography, immunoaffinity chromatography, etc.

Laminin-5 is described in detail in JP 2001-172196 A, which is incorporated herein by reference.

Laminin is structured as a heterotrimer molecule composed of α, β and γ chains linked to each other via disulfide linkages, which takes a characteristic cross-structure. Each chain is composed of two or more domains, and domains I and II form a triple helix. Before the filing of the present application, at least 15 isoforms of laminin molecules have been identified from different combinations of 5 types of α chains (α1 to α5), 3 types of β chains (β1 to β3) and 3 types of γ chains (γ1 to γ3), and it is suggested that there are actually several times that number of isoforms. Laminin 1, which is a typical laminin, is a hetero trimer molecule composed of α1, β1, γ1, and laminin-5 used in the present application is composed of α3, β3, γ2. For example, when the homology of the polypeptide chains of laminin-1 and laminin-5 are analyzed using software such as Genetyx, the homology of α1 and α3 is 42%, that of β1 and β3 is 41%, and that of γ1 and γ2 is 54%. Although laminin-1 and laminin-5 are both laminins, they are presumed to show different characteristics, because they respectively have compositions, (α1, β1, γ1) and (α3, β3, γ2), consisting of α, β, γ chains, wherein each of the three chains are encoded by a completely different gene than that of the other laminin, and further, even in the same a chain, α1 and α3, encoded by two different genes has a homology of merely 42%.

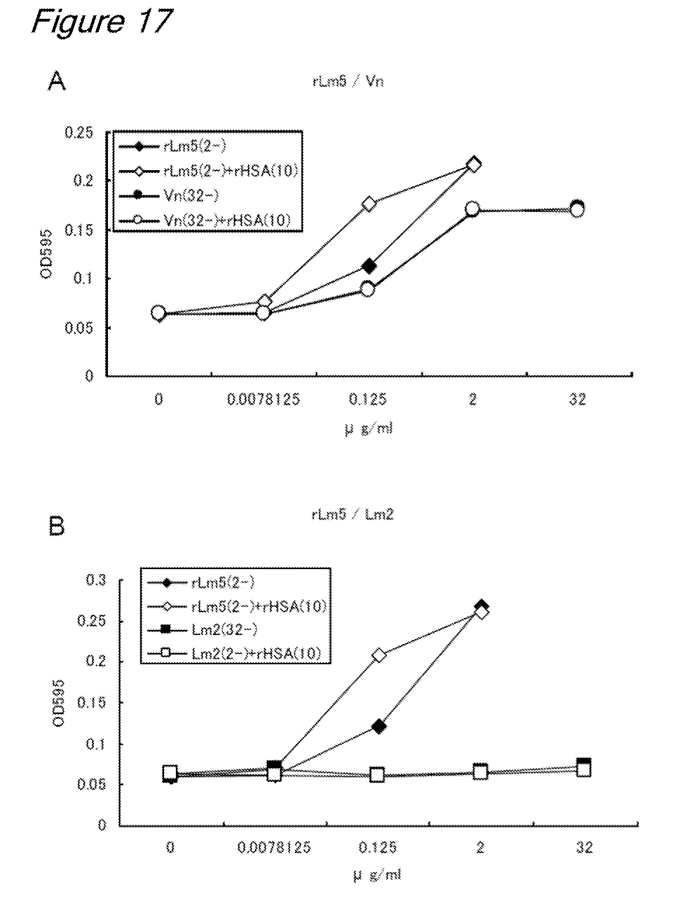

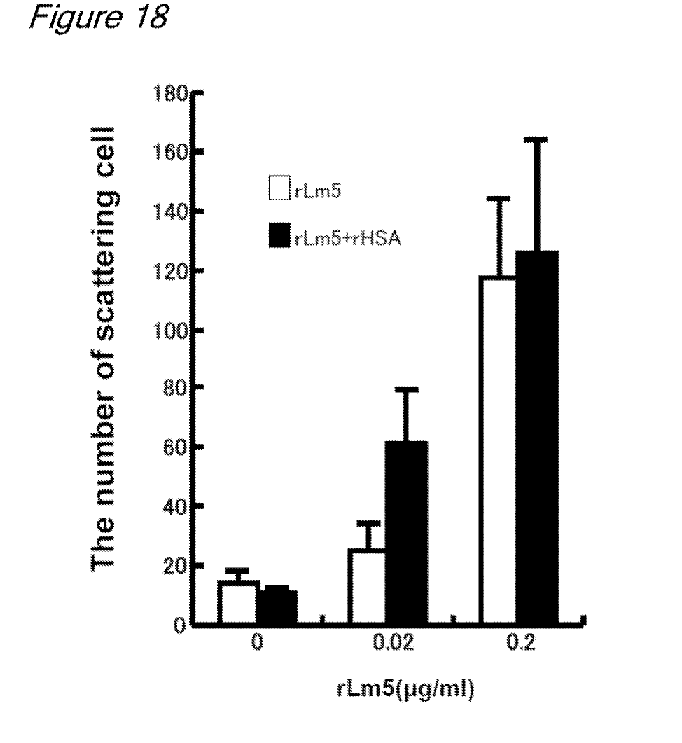

As shown in Example 3, no activity elevating effect was exhibited for vitronectin, which is another extracellular matrix, and laminin-2, which is another laminin isoform, as shown in Example 3. (FIG. 17) As shown above, the activity elevating effect is not a phenomenon common to all extracellular matrix proteins, and further, it is not a phenomenon common to any isoform of laminin.

Polypeptide

The present invention is characterized by the increase in the various activities of Lm5 in cell culture in combination with a specific polypeptide in a cell culturing system containing Lm5.

Polypeptide is selected from a group consisting of: a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone.

1) Protein in blood

The present invention preferably uses protein in blood, more preferably protein in blood other than the extracellular matrix protein, with laminin-5 protein.

The protein in blood is preferably selected from a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin. These are all proteins in blood other than the extracellular matrix protein.

An “extracellular matrix” is a substance filling the extracellular space. At the same time, it acts as a bone structure (e.g. cartilage or bone of an animal), a foothold for cell adhesion (e.g. basement membranes or fibronectin), and a retainer and a provider of cell growth factor (e.g. a cell growth factor binding to heparan sulfate, i.e. FGF). It may be said that many cells constituting multicellular organisms live hidden in a bed or a nest of the extracellular matrix. Essential ingredients in the extracellular matrix of vertebrates including human are glycoprotein such as collagen, proteoglycan, fibronectin or laminin (some are cell adhesion molecules). An “extracellular matrix protein” is a protein constituting the above extracellular matrix.

The “protein in blood other than the extracellular matrix protein” in the present invention is those proteins in blood that are not extracellular matrix proteins related to cell adhesion and other activities. These are all proteins known in the art that a person skilled in the art can obtain as necessary.

The “protein in blood other than extracellular matrix protein” is not limited, but it is preferably human serum albumin (HSA/obtainable, for example, from Nacalai), recombinant human serum albumin (rHSA/obtainable, for example, from SIGMA), or bovine serum albumin (BSA/obtainable, for example, from SIGMA).

The “protein in blood other than extracellular matrix protein” may be immunoglobulin. Immunoglobulin is well known by a person skilled in the art, and includes IgG, IgA, IgM, IgD, IgE. A human immunoglobulin (IgG/e.g. obtainable from Oriental Yeast Co., Ltd.) may be used, for example.

2) Gelatin

Gelatin is extracted from collagen, which is the main ingredient of connective tissues, such as the skin, the bones, and the tendon of an animal, by heating the collagen. The main ingredient of gelatin is protein.

Protein in the Tumor Necrosis Factor (TNF) Family

“Tumor Necrosis Factor (TNF)” is a type of cytokine, consisting of the following three types by narrow definition: TNF-α, TNF-β (lymphotoxin (LT)-α) and LT-β. The “protein in the TNF family” includes at least 19 types of molecules including receptor activating factor NFkB ligand (RANKL), Fas ligand, CD40 ligand.

Receptor activating factor NFkB ligand (RANKL) can be preferably used as an example of “protein in the TNF family” of the present invention.

4) Peptone

A “peptone” is protein digested by protease. Protein is digested in the stomach by pepsin to become peptone, and then peptone is further digested to amino acid by the pancreatic juice secreted from the pancreas and the intestinal juice secreted from jejunum.

Peptone is often added to the culture, since it is a suitable nutrition source of microorganisms. The peptone acting as a nutrition source in the culture is protein hydrolysized to amino acid and peptide with low molecular weight, commonly using an enzymolysis product (e.g. protease such as pancreatin derived from the pancreas of a swine) of milk protein (milk casein).

Any peptone can be used, but it is preferable to use a plant derived peptone. It is selected, for example, from a group consisting of a cotton seed-derived peptone, a soy bean-derived peptone, a wheat-derived peptone and a pea-derived peptone.

The effect of the present invention to increase the activity of laminin-5 was not obtained from a simple amino acid as shown in Example 1. Accordingly, the “peptide” of the present invention does not include a peptone digested to a simple amino acid.

Cells

The types and origins of cells to be cultured in the method of the present invention are not particularly limited.

Cells are preferably selected from a group consisting of pluripotent stem cells, tissue stem cells, somatic cells, germ cells and sacroma cells. Any pluripotent stem cells can be used, but they are preferably selected from embryonic stem cells, induced pluripotent stem cells, embryonic germ cells or germline stem cells. Tissue stem cells are preferably selected from mesenchymal stem cells, hepatic stem cells, pancreatic stem cells, neural stem cells, skin stem cells or hematopoietic stem cells. The somatic cells are preferably selected from hepatic cells, pancreatic cells, muscle cells, osteocytes, osteoblasts, osteoclasts, cartilage cells, fat cells, skin cells, fibroblasts, pancreatic cells, kidney cells, pneumocytes or blood cells, which are lymphocites, red blood cells, white blood cells, monocytes, macrophage or megakaryocytes.

Species of organisms acting as the origin of the cells are not particularly limited either. Cells are preferably derived from mammals such as mice, rats, humans, monkeys, pigs, dogs, sheep and goats and birds such as chickens. More preferably, they are derived from the species selected from a group consisting of mice, rats and humans.

As used herein, the term “pluripotent stem cells” is intended to collectively refer to stem cells capable of differentiating into cells of any tissue type (pluripotency). Although ES cells (EB3 cells) are used for study in the Examples below, pluripotent stem cells that can be used in the method of the present invention include not only embryonic stem cells, but also all pluripotent stem cells derived from, e.g., cells of adult mammalian organs or tissues, bone marrow cells, blood cells, and embryonic or fetal cells, as long as their characteristics are similar to those of embryonic stem cells. Characteristics similar to embryonic stem cells in the present context can be defined as a cytobiological features specific to embryonic stem cells, which are a gene expression specific to embryonic stem cells and differentiation capability to all germ layers, such as endoderm, mesoderm, and ectoderm.

Specific examples of cells that can be proliferated by the method of the present invention include, but are not limited to, embryonic stem cells (ES cells), induced pluripotent stem cells (iPS cells), embryonic germ cells (EG cells), germline stem cells (GS cells) and so on. It should be noted that pluripotent stem cells preferred in the present invention are ES cells and iPS cells. An iPS cell is especially preferable for reasons including that it poses no ethical problem. Any known pluripotent stem cell can be used. An example is the pluripotent stem cells described in International Publication WO 2009/123349 (PCT/JP 2009/057041).

“Tissue stem cells” are stem cells that can be differentiated to various cell species (pluripotency), although only cell lines of specific tissues are differentiable. For example, hematopoietic stem cells in the bone marrow form blood and neural stem cells differentiate into nerve cells. A variety of other cells are included, such as hepatic stem cells to create the liver, and skin stem cells to create skin tissue.

“Somatic cells” are cells, other than germ cells, constituting the multicellular organisms. These cells are not inherited to the next generation in a sexual reproduction. The term in the present specification represents various cells other than the “pluripotent stem cells” and “tissue stem cells”.

System Comprising laminin-5

The present invention cultures cells in a system containing laminin-5. The terms “system containing laminin-5” in the present invention means inclution of laminin-5 in the cell culture system in one form or another, without any limitation to the embodiment.

An embodiment of using a culture vessel treated by laminin-5, especially coated by laminin-5, is preferable for culturing cells in a system containing laminin-5 in the present invention.

A “cell culture vessel” is not particularly limited in the present invention, and a vessel sterilized to prevent contamination by germs, made of any material and any shape suitable for culturing cells can be used. Examples of such culture vessel include a culture dish, a culture flask, a culture schale, a culture plate with 96 wells, 48 wells, 12 wells, 6 wells or 4 wells and so on, and a culture bottle, which are commonly used in the present field of art, without being limited thereby.

The present invention is characterized by using laminin-5 and polypeptide in cell culture. Preferably, the surface of the cell culture vessel is treated by immobilizing (coating) laminin-5 and polypeptide. Treating the culture vessel by immobilizing laminin-5 on its surface is a treatment technique known in the art, and a person skilled in the art may adopt any culture vessel depending on the purpose of the present invention to treat the vessel with laminin-5 and polypeptide and use the treated vessel to cultured cells by the method of the present invention.

The amount of laminin-5 used in the treatment of the cell culture vessel is not particularly limited. A good result is obtained when the vessel is treated with a solution containing preferably 0.01 μg/ml or more, preferably 0.1-15 μg/ml, and more preferably 0.1 g/ml-2 μg/ml of laminin-5.

A treatment of the culture vessel by laminin-5 in an embodiment of the present invention may comprise drying or other treatments after depositing laminin-5 on the inner surface of the culture vessel. A culture medium commonly used for cell culture, such as GMEM (GIBCO) and DMEM, may be placed in a culture vessel treated with laminin-5, and pluripotent stem cells are added to that culture medium. Then, cells are cultured under appropriate culture conditions known in the art, such as, at 37° C., in 5% carbon dioxide air layer, without being limited thereby.

It is preferable in the present invention to treat (coat) the cell culture vessel with laminin-5 and polypeptide. The order of treating a cell culture vessel is not particularly limited, but preferably, it is treated with polypeptide before it is treated with laminin, or it is treated with polypeptide and laminin at the same time.

Accordingly, the present invention also provides compositions for coating the cell culture vessel or a cell culture vessel coating agent comprising the aforementioned polypeptide. The composition or agent of the present invention may contain laminin-5 in combination with the polypeptide. The present invention further provides a kit comprising the cell culture medium containing the above mentioned polypeptide. The present invention also provides a kit comprising the cell culture medium containing polypeptide and laminin-5 aforementioned. The kit of the present invention may also include a precoated culture dish, a precoated culture plate or the like other than the cell culture medium.

The composition, the agent or the kit of the present invention can be used in a method for culturing cells in a system containing laminin-5.

Amount of Polypeptide to be Used

The amount of Polypeptide to be used is not particularly limited in the present invention. A person skilled in the art can appropriately select an appropriate amount depending on elements such as the type of polypeptide to be used.

Polypeptide is preferably used at a concentration of 1 μg/ml to 200 μg/ml, without being limited thereby. Example 1 showed that a concentration of 3.125 μg/ml to 12.5 μg/ml is especially preferable when protein in blood, such as HSA, BSA, HS or IgG is used. Further, when a cotton seed derived peptone was used, the cell activity elevating effect against laminin-5 was exhibited at higher concentrations, preferably 15.6 g/ml to 1000 μg/ml.

Combined Use of Polypeptide

In a preferable embodiment of the present invention, two or more types of polypeptide are included in the cell culturing system. When rHSA and IgG were combined for use as polypeptide in Example 1, an adhesive activity as strong as that of using 10 μg/ml of rHSA was obtained, even though each was used at a low concentration (0.25 μg/ml) that would not provide any large effect alone. Accordingly, it is considered that the combined use of two or more types of polypeptides provides a synergistic effect, not an additive effect.

Effects of the Present Invention

Various activities of laminin-5 protein induced in the cell culture of the present invention increase when laminin-5 is combined for use with polypeptide. The effects of laminin-5 include a cell adhesion activity, a cell scattering activity, a wound healing activity, a proliferation stimulating activity, an activity for maintaining undifferentiated-state and an activity for maintaining pluripotency, without being limited thereby.

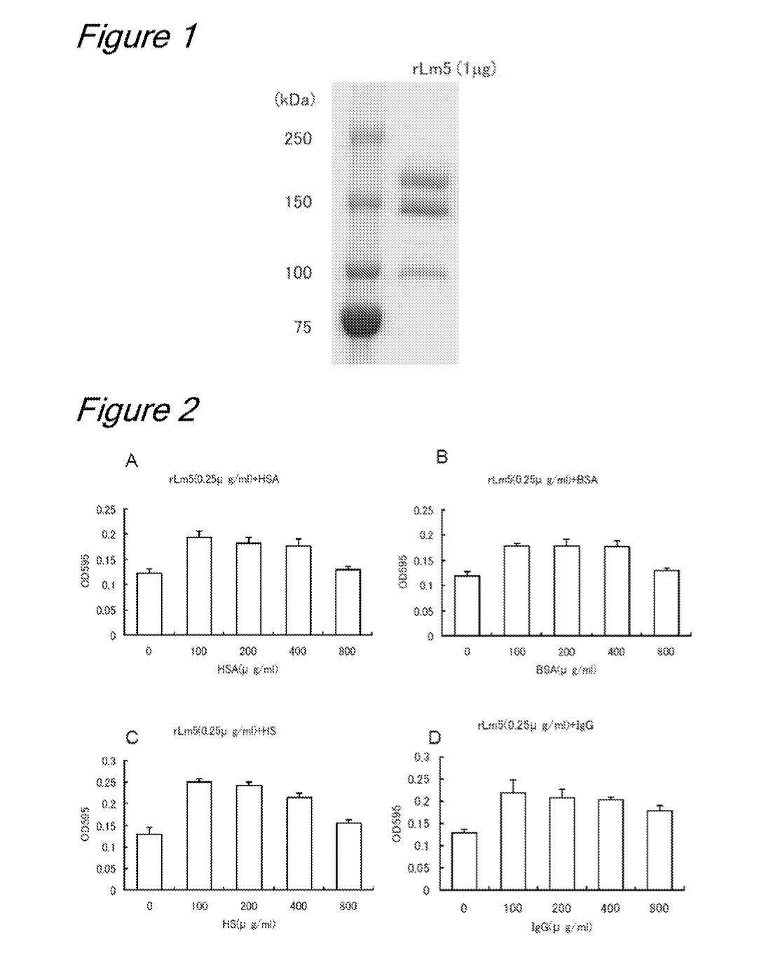

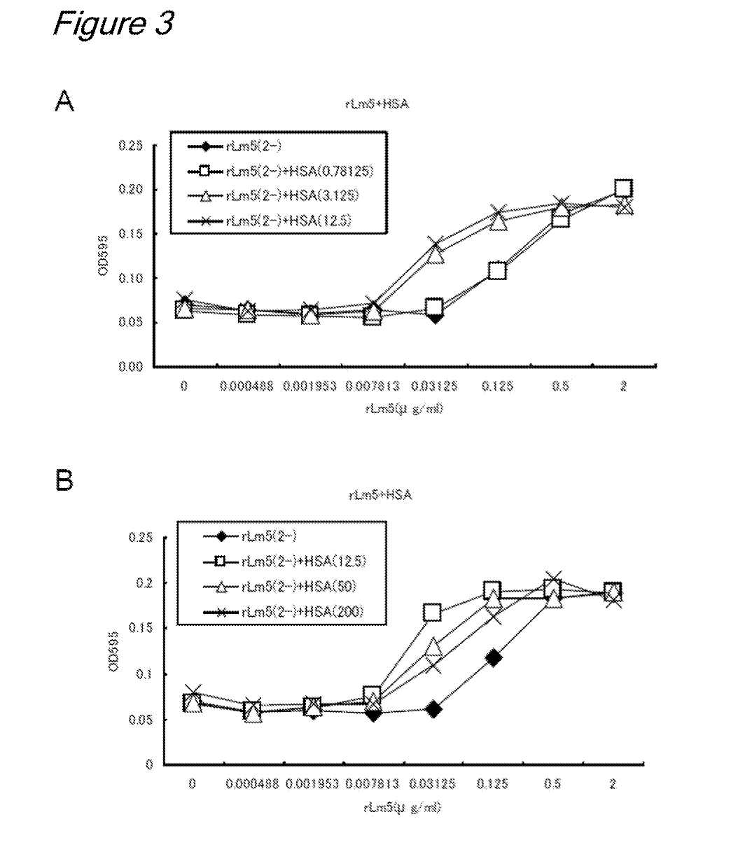

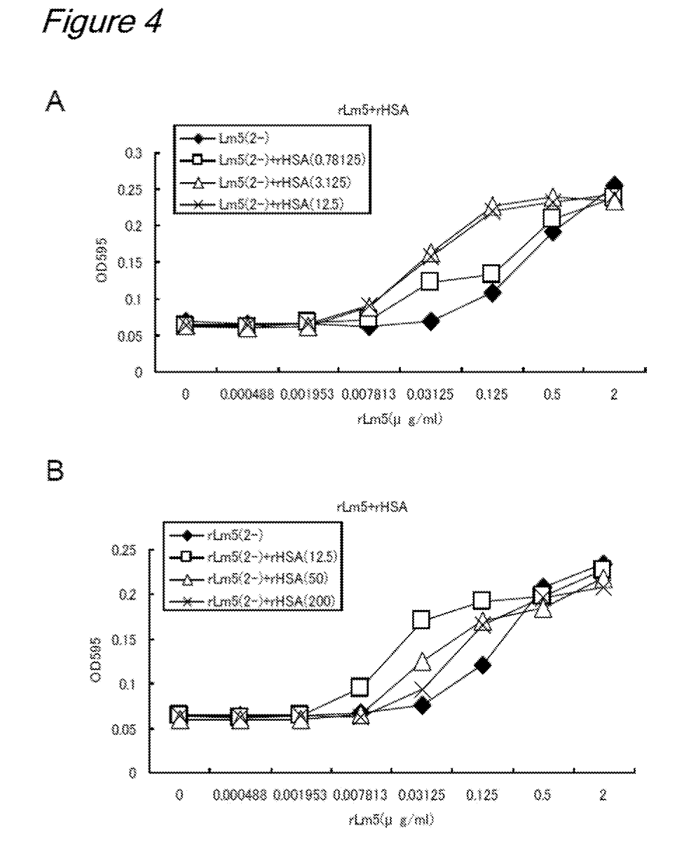

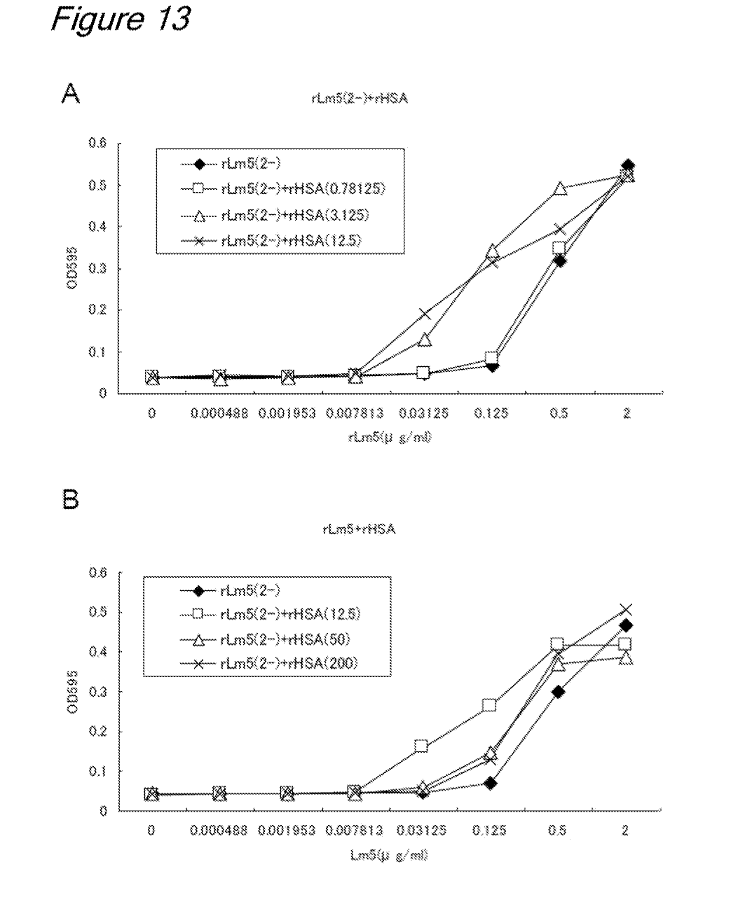

A “cell adhesion activity” is an effect of inducing cell adhesion. In FIG. 2, FIG. 3 and FIG. 4, a combined use of HSA with rLm5 (0.125 μg/ml) produced an adhesion activity equivalent to that of rLm5 (2 μg/ml). Likewise, in FIG. 13, a combined used of HSA with rLm5 (0.5 μg/ml) produced an adhesion activity equivalent to that of rLm5 (2 μg/ml). Hence, the use of polypeptide increases the cell adhesion activity of the present invention to preferably 1.2 times or more, more preferably 4 times or more, most preferably 8 times or more the activity of an invention not using polypeptide.

A “cell scattering activity” is an effect of scattering cells. The use of polypeptide increases the cell scattering activity of the present invention to preferably twice the activity of an invention not using polypeptide.

A “wound healing activity” is an effect of healing wounds. In other words, it is an activity brought about by the coating of, for example, laminin-5 on a physically injured section abandoned by the cells, resulting in the migration of surrounding cells to the coated section. The wound healing effect can be determined by assessing the healing ratio through measuring the wound width after specific periods from the time the subject has been wounded (e.g. after 16 hours). The combined use of polypeptide (rHSA) with human laminin-5 in Example 5 increased the wound healing percentage from 60% to 80%.

A “cell proliferation activity” is an effect of advancing cell proliferation. The effect of cell proliferation can be determined by measuring the number of cells after a specific length of time from the start of cell culture.

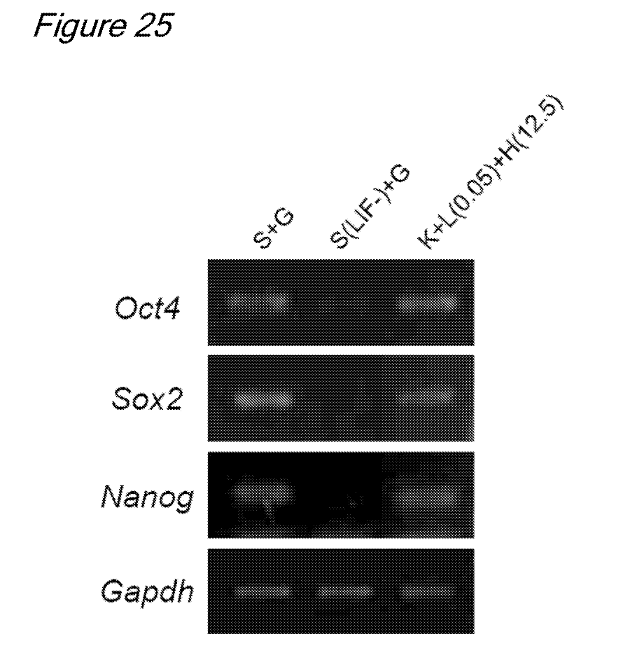

An “activity for maintaining undifferentiated-state” is the effect of maintaining the undifferentiated state when the cells to be cultured are undifferentiated cells, such as, pluripotent stem cells or tissue stem cells. Culturing the cells with laiminin-5 keeps the cells from being differentiated and maintains the undifferentiated state. The undifferentiated state is also maintained when laminin-5 and polypeptide are combined for use (Example 7, FIG. 25). Sox2, Nanog, Oct4 and other undifferentiated-state markers can be measured to confirm whether the undifferentiated state is maintained during culture.

An “activity maintaining pluripotency” is the act of maintaining pluripotency when the cells to be cultured have pluripotency, for example, when they are pluripotent stem cells. Pluripotency is also maintained when laminin-5 and polypeptide are combined for use in the present invention.

### EXAMPLES

The present invention will now be described in more detail below on the basis of the following examples, which are not intended to limit the scope of the invention.

### Example 1

### Preparation of Recombinant Human laminin-5 (rLm5)

In this example, a recombinant human laminin-5 protein was prepared in a known manner.

From human fetal kidney cell line HEK293 modified to carry cDNAs for α3 chain (SEQ ID NO: 1), β3 chain (SEQ ID NO: 3) and γ2 chain (SEQ ID NO: 5) (Lm5-HEK293), the serum-free supernatant was collected and centrifuged at 4° C. at 3000 rpm for 5 minutes. The human fetal kidney cell line HEK293 was obtained as described in J. Biochem. 132:607-612 (2002). The supernatant was then applied to Heparin sepharose CL-6B (GE healthcare) and eluted. The rLm5-containing fractions were passed through an antibody column, in which mouse anti-Lm-α3 (anti-laminin α3) monoclonal antibody (BG5) was covalently bonded to Protein A sepharose CL-6B (GE healthcare), and then eluted. It should be noted that monoclonal antibody BG5 is an antibody prepared by the inventors of the present invention using an N terminal fragment of the laminin α3B chain as an antigen according to known procedures for monoclonal antibody preparation.

Purified rLm5 (1 μg) was denaturated under reducing conditions and then subjected to SDS polyacrylamide gel electrophoresis on a 5-20% gel to confirm the size and purity of α3, β3 and γ2 chains. As a result, bands of 160 kDa, 135 kDa and 105 kDa were detected, respectively. FIG. 1 shows a photograph of SDS polyacrylamide gel electrophoresis obtained for purified rLm5. When analyzed with a CS-Analyzer, purified rLm5 was found to have a purity of about 98%. rLm5 thus prepared was used in the following examples.

### Example 2

### Cell Adhesion Assay

This example shows the result of an adhesion assay when rLm5 is added to various cells and when additives are added in addition to rLm5.

Four types of cells, specifically, rat hepatic cell line (BRL), mouse ES cell line (EB3), human sarcoma cell line (HT1080), human mesenchymal stem cell (hMSC), were used. BRL was offered by Yokohama City University, Graduate School of Nanobioscience, Department of Genome System Science. EB3 was offered by Osaka University, Graduate School of Medicine, Frontier Biosciences G6, Course on Molecular Treatment, Field of Stem Cell Regulation. HT1080 was obtained from Riken BioResource Center (RCB 1956). hMSC was obtained from Lonza Corporation.

Different types of cells were cultured and proliferated in the following culture media: BRL in DMEM/F12 with 10% fetal bovine serum (FBS) added to it; EB3 in GMEM (GIBCO) with 10% FBS, 0.1 mM of non-essential amino acid (Gibco), 1 mM of sodium pyruvate (Gibco), 1000 U/ml of ESGRO (Millipore), and 10⁻⁴ M of 2-mercaptoethanol (WAKO); HT1080 in MEM (SIGMA) with 10% FBS added to it; and hMSC in MSCGM (LONZA). A serum-free medium, that is, media wherein serum has been removed, were used to perform the adhesion assay.

A 96-well plate (Corning) was treated with a concentration-controlled rLm5 at 37° C. for 2 hours or at 4° C. overnight, then the treated surface was washed with PBS(−), and was subjected to 1 hour of blocking treatment at 37° C. The rLm5 used in the treatment was mixed with human serum albumin (HSA/Nacalai), recombinant human serum albumin (rHSA/SIGMA), human serum (HS/OYC), bovine serum albumin (BSA/SIGMA), human immunoglobulin (IgG/OYC), bovine gelatin (Gl/SIGMA), recombinant human Receptor Activator of NF-κB Ligand (sRANKL/OYC), cotton seed-derived peptone (Pep/DMV), glycine (Gly/Nacalai), arginine (Arg/Nacalai), trehalose (Tre/SIGMA), xylose (Xyl/Wako), mannose (Man/Wako), lactose (Lac/Wako) as necessary. The cells were washed in a serum-free medium, i.e., media to which no serum was added, then, they were seeded in the well at 20000 cells/well, and cultured for 1 hour at 37° C., under an air layer of 5% CO₂ and 95% air. However, EB3 was seeded in the well at 30000 cells/well. After culturing completed, the plates were lightly shaken by the Vortex mixer to let cells with weak adhesion float off to be removed by Percoll (GE healthcare) treatment. The adhered cells were fixed by 25% glutaraldehyde (Nacalai), dyed with 2.5% crystal violet (Nacalai), and measured with OD595 to assess the adhesion activity of rLm5 at various conditions.

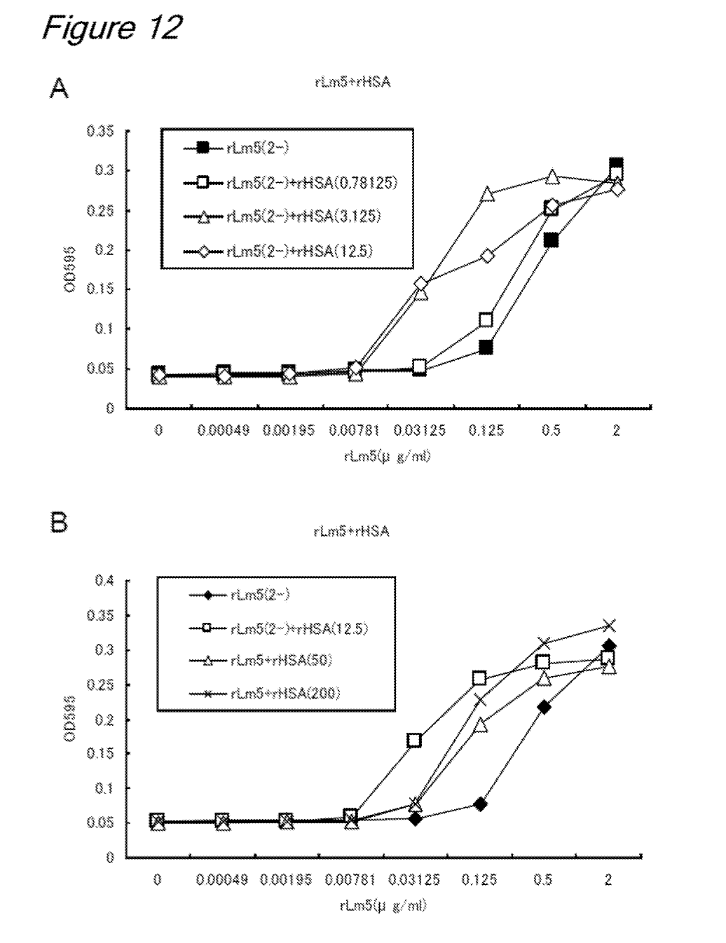

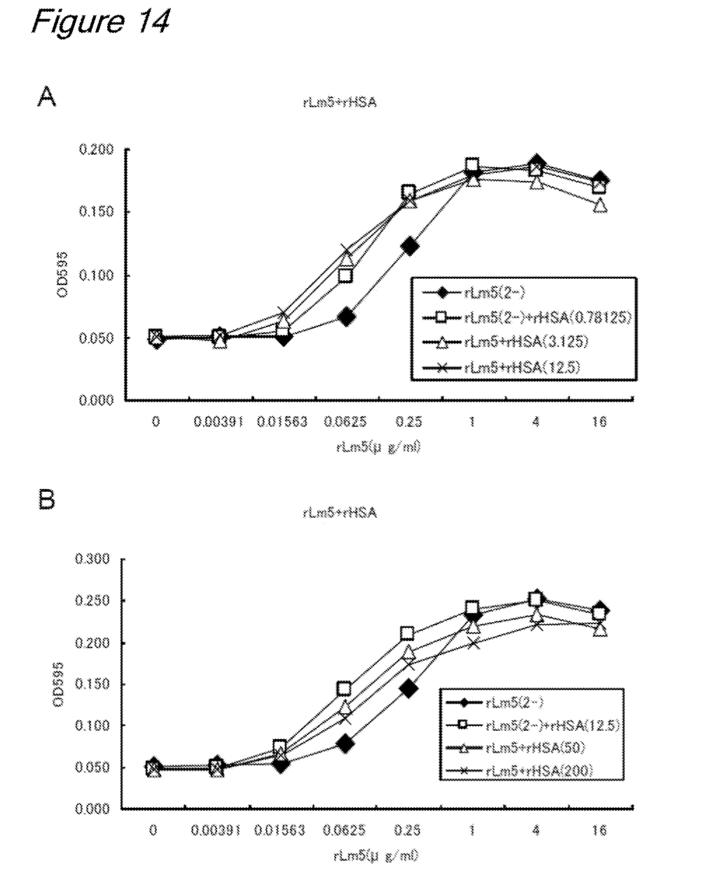

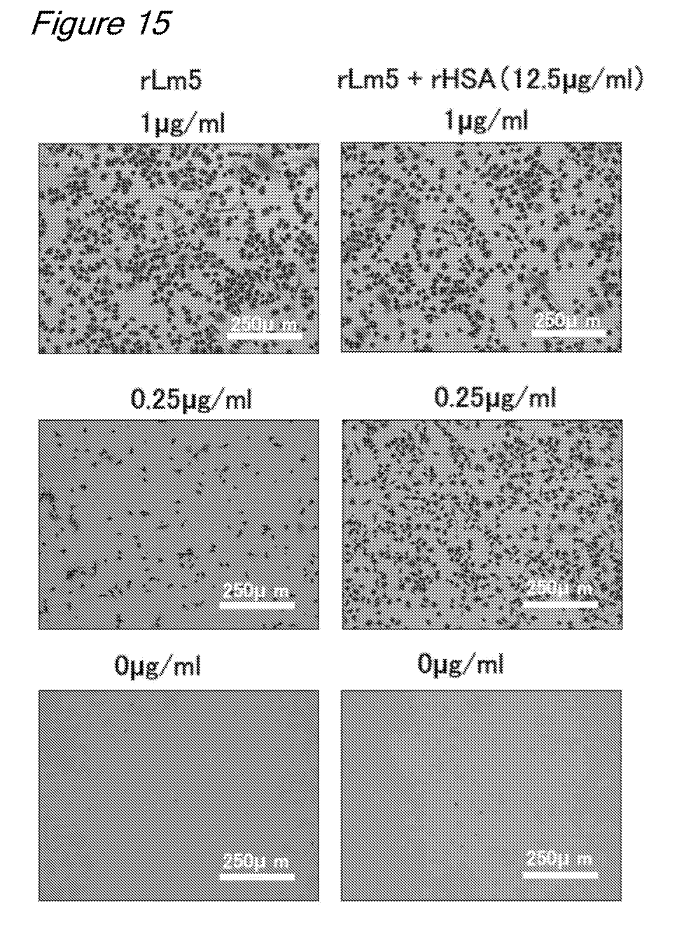

FIGS. 2-11 show the result of the adhesion assay using BRL; FIG. 12 shows the result of the adhesion assay using HT1080; FIG. 13 shows the result of the adhesion assay using hMSC; and FIGS. 14, 15 show the result of the adhesion assay using EB3. In a treatment with 0.25 g/ml of rLm5, the adhesion activity was higher when HSA, BSA, HS, IgG were combined for use than when they were not. This result showed that protein in blood is effective in increasing the adhesion activity of rLm5 (FIG. 2). HSA (0-200 μg/ml) were combined for use to determine the optimum concentration of HSA. The result was that the optimum concentration is in the range of 3.125-12.5 g/ml (FIG. 3). Further, 0-200 μg/ml of rHSA was combined for use to identify the substance showing the activity elevating effect. The result showed that rHSA has an optimum concentration in the range of 3.125-12.5 μg/ml, like HSA. It was thus concluded that the activity elevating effect is not exhibited by substances such as impurities in the naturally occurring protein (FIG. 4).

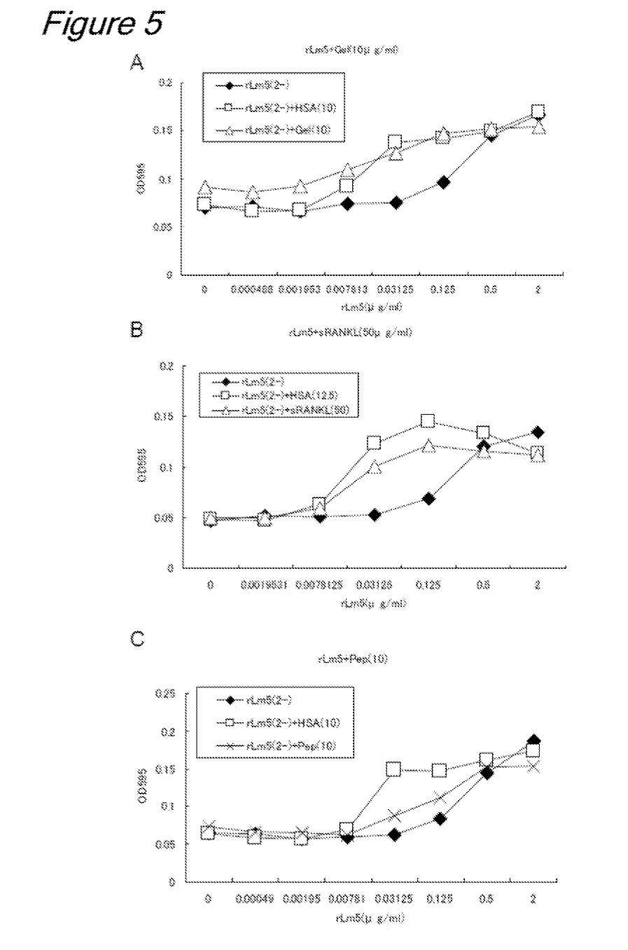

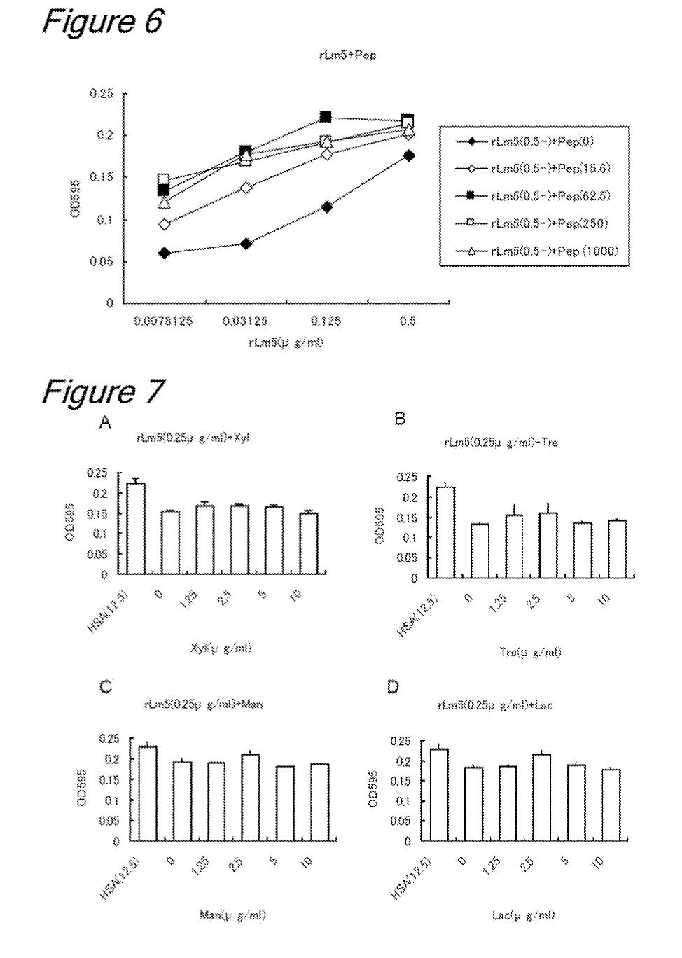

Then, Gl, sRANKL, Pep were used to determine whether proteins other than proteins in blood, such as HSA, BSA, HS, IgG, have the same effects. The result was that the effects differed slightly by the substance, but they commonly had adhesion activity elevating effects like HSA (FIG. 5). Although the activity elevating effect of Pep was minute, an evaluation performed at a wider range of concentrations (0-1000 μg/ml) showed that Pep exhibits a strong activity elevating effect at high concentrations (FIG. 6). From the above observations, it was concluded that polypeptide or peptide would exhibit an activity elevating effect.

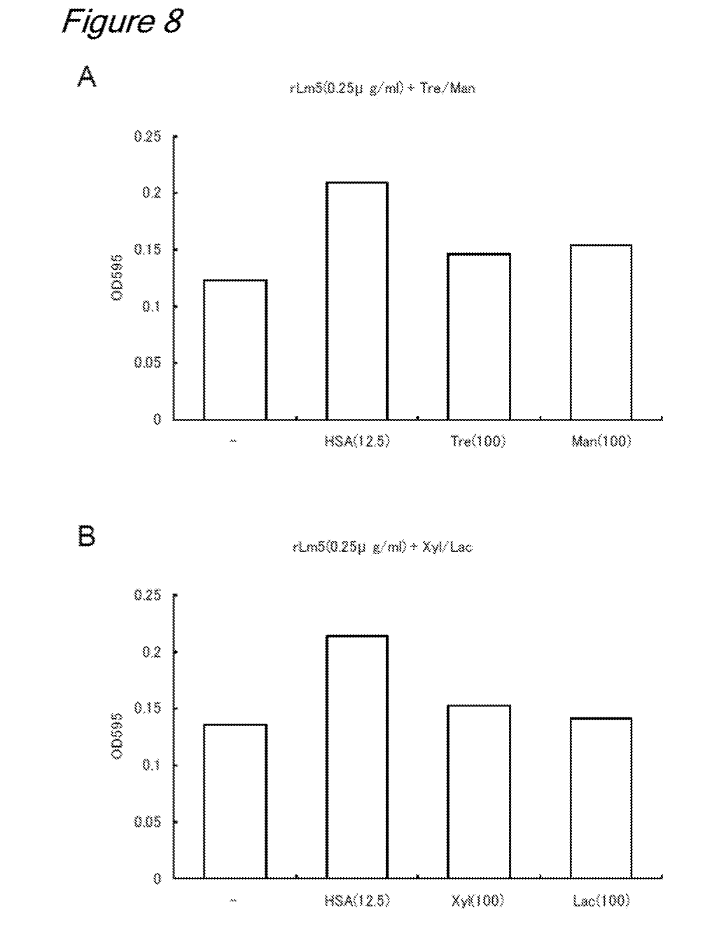

Sugars were assessed next. A combined use of Xyl, Man, Lac or Tre with 0.25 μg/ml of rLm5 did not produce an activity elevating effect like that of the combined use of polypeptide or peptide (FIG. 7). A combined use at a higher concentration (100 μg/ml) did not present a strong activity elevating effect like HSA (FIG. 8).

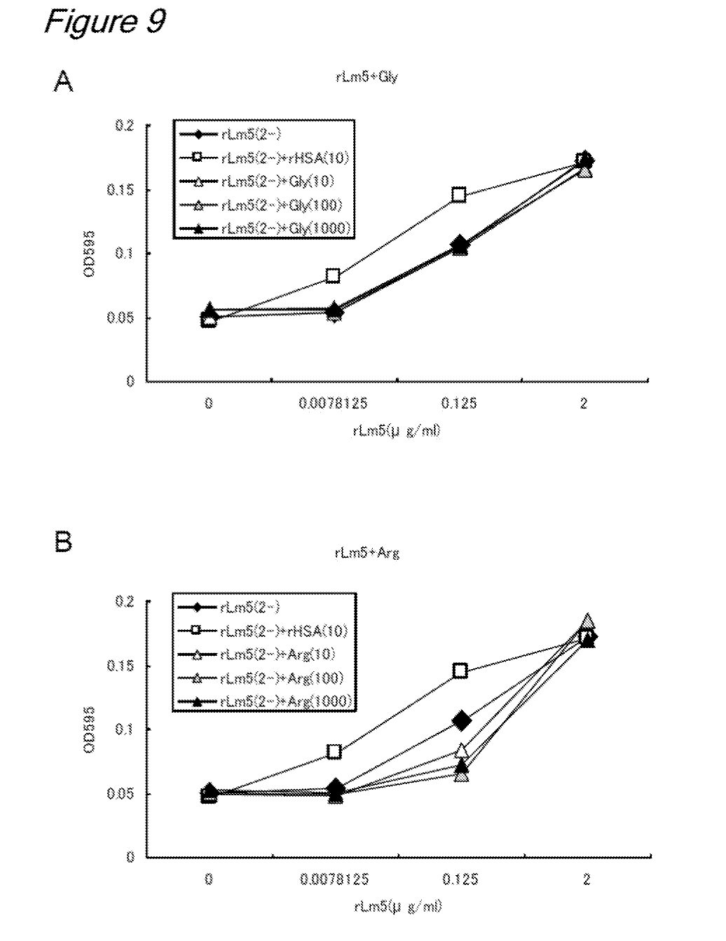

Amino acids were assessed next. They were similar to sugars in that a combined use of 0-1000 μg/ml of Gly or Arg with rLm5 did not provide a strong activity elevating effect like that of rHSA (FIG. 9). The activity decreased for Arg.

The above results show that activity elevating effects are not exhibited by low molecules such as sugars and amino acids.

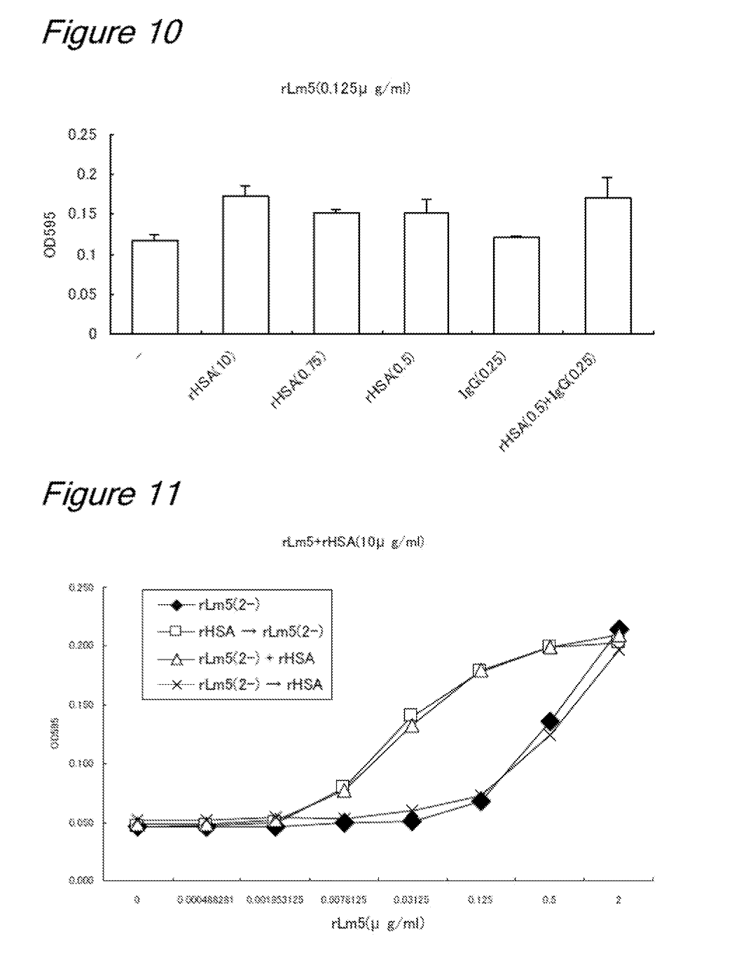

A combined use of multiple proteins was assessed next. The adhesion activity was low when either 0.5 g/ml of rHSA alone or 0.25 μg/ml of IgG alone was combined for use with 0.125 μg/ml of rLm5 relative to when 10 μg/ml of rHSA was combined for use with the same rLm5. However, when 0.5 μg/ml of rHSA and 0.25 μg/ml of IgG were both combined for use with rLm5, the adhesion activity was equivalent to that of using 10 μg/ml of rHSA (FIG. 10). The result showed that a combination of multiple types of proteins increases the adhesion activity of rLm5.

The combination method was subsequently assessed using 10 μg/ml of rHSA. The following cases were compared: a case of treating with rHSA before treating with rLm5 (rHSA→rLm5); the conventional method of treating with rHSA and rLM5 at the same time (rLm5+rHSA); and treating with rLm5 before treating with rHSA (rLm5→rHSA). The result showed that the use of rHSA in the preliminary treatment or together with rLm5 provides adhesion activity elevating effects, but the use of rHSA in the later treatment does not provide adhesion activity elevating effects (FIG. 11).

Next, HT1080, hMSC, EB3 were used to assess whether activity elevating effects like those in BRL are exhibited in cells other than BRL. The assessment was conducted with 0-200 μg/ml of rHSA used in combination with rLm5. The result was that activity elevating effects were seen in all cells, although there were some differences in the activity elevating effects according to the cell type. Further, optimum concentration was exhibited is in the range of 3.125-12.5 μg/ml like BRL (FIGS. 12-14). The morphology of EB3 cells after adhesion assay is shown in FIG. 15.

The results showed that performing treatments by combining polypeptide or peptide with rLm5 will raise the adhesion activity of rLm5 in various cells. Further, the optimum concentration of polypeptide combined for use is 3.125-12.5 μg/ml, and the polypeptide or peptide to be combined does not have to be used at the same time as rLm5, but it can be used in advance and still increase the rLm5 activity sufficiently.

### Example 3

### Assessment of Activity Elevating Effects of Other Extracellular Matrix Proteins or Laminin Isoforms

This example shows the result of an assessment by an adhesion assay using rat hepatic cell line (BRL) to consider whether an activity elevating effect similar to that of rLm5 is exhibited in other extracellular matrix proteins or laminin isoforms, such as human vitronectin (Vn/SIGMA) and human laminin 2 (Lm2/Millipore). The assay was conducted in accordance with the method described in Example 2.

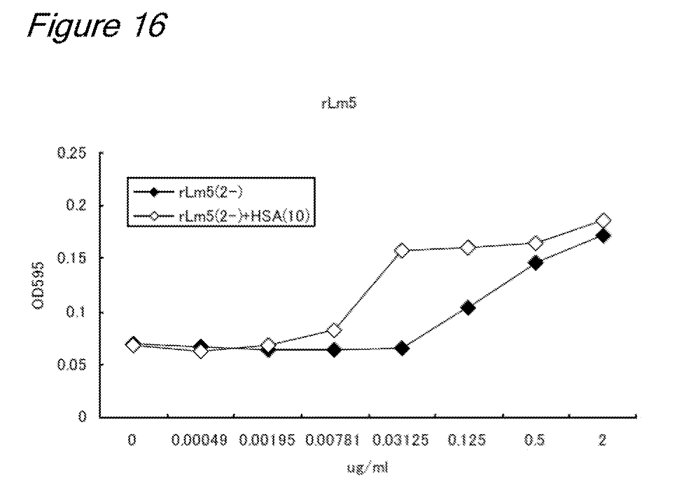

FIG. 16 and FIG. 17 respectively show the result of an adhesion assay in which 10 μg/ml of HSA and 10 μg/ml of rHSA are combined. Activity elevating effects from combination with HSA or rHSA, as exhibited for rLm5, were not exhibited at all for Vn and Lm2. Such result indicates that the activity elevating effect is not a phenomenon occurring to any extracellular matrix protein, moreover, the phenomenon is not common to all isoforms of laminin.

### Example 4

### Cell Scattering Assay Using BRL Cells

This example shows the result of a cell scattering assay when rLm5 is added to rat hepatic cell line (BRL) cells and when additives are added in addition to rLm5. BRL was offered by Yokohama City University, Graduate School of Nanobioscience, Department of Genome System Science.

BRL cells were cultured and proliferated in DMEM/F12 with 10% fetal bovine serum (FBS) added to it. However, the cell scattering assay was conducted using DMEM/F12 culture with 1% FBS added to it.

A 24-well plate (Nunc) was treated with a concentration-controlled rLm5 at 4° C. overnight, then the treated surface was washed with PBS(−), and t was subjected to 1 hour of blocking treatment in a 1% BSA (SIGMA) solution at 37° C. The rLm5 used in the treatment was mixed with rHSA as necessary. After washing the rLm5-treated surface with PBS(−), the cells were washed in a 1% FBS culture, then they were seeded in the well at 7000 cells/well and subsequently cultured for 40 hours at 37° C., under an air layer of 5% CO₂ and 95% air. After culturing completed, the plates were lightly shaken by the Vortex mixer to let cells with weak adhesion float off to be removed by Percoll treatment. The adhered cells were fixed by 25% glutaraldehyde. Photographs of 3 random fields were taken to count the number of single cell.

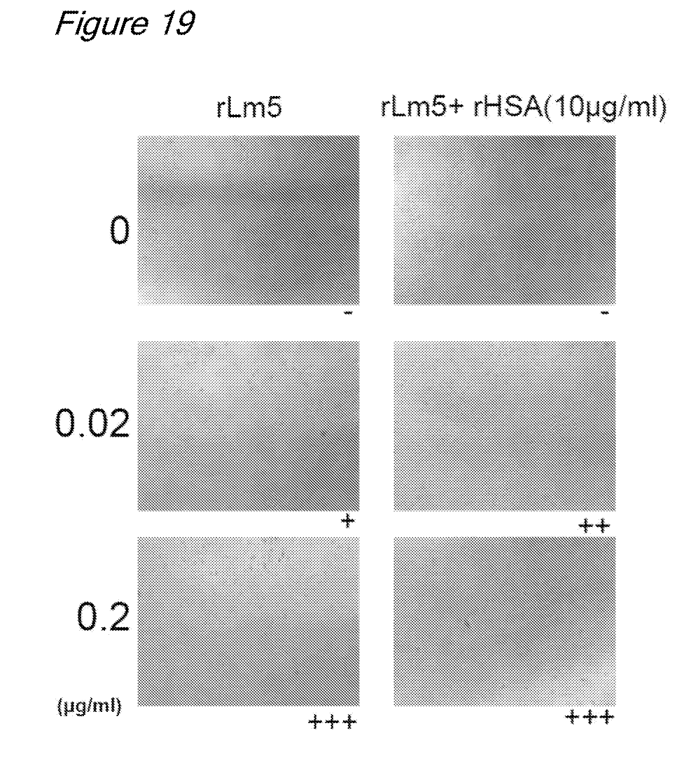

The result of the cell scattering assay is shown in FIGS. 18 and 19. A significant increase in the cell scattering activity was recognized when rHSA was added to Lm5 of a low concentration (0.02 μg/ml).

The result indicates that an activity elevating effect of combining rHSA and other polypeptides for use is recognized not only for the cell adhesion activity, but also for the cell scattering activity, which is already reported as an activity of rLm5.

### Example 5

### Wound Healing Assay Using BRL Cells

This example shows the result of a wound healing assay when rLm5 is added to rat hepatic cell line (BRL) cells and when additives are added in addition to rLm5. BRL was offered by Yokohama City University, Graduate School of Nanobioscience, Department of Genome System Science.

BRL cells were cultured and proliferated in DMEM/F12 with 10% fetal bovine serum (FBS) added to it. However, the wound healing assay was conducted using medium with serum-free medium, having no serum therein, added to the medium.

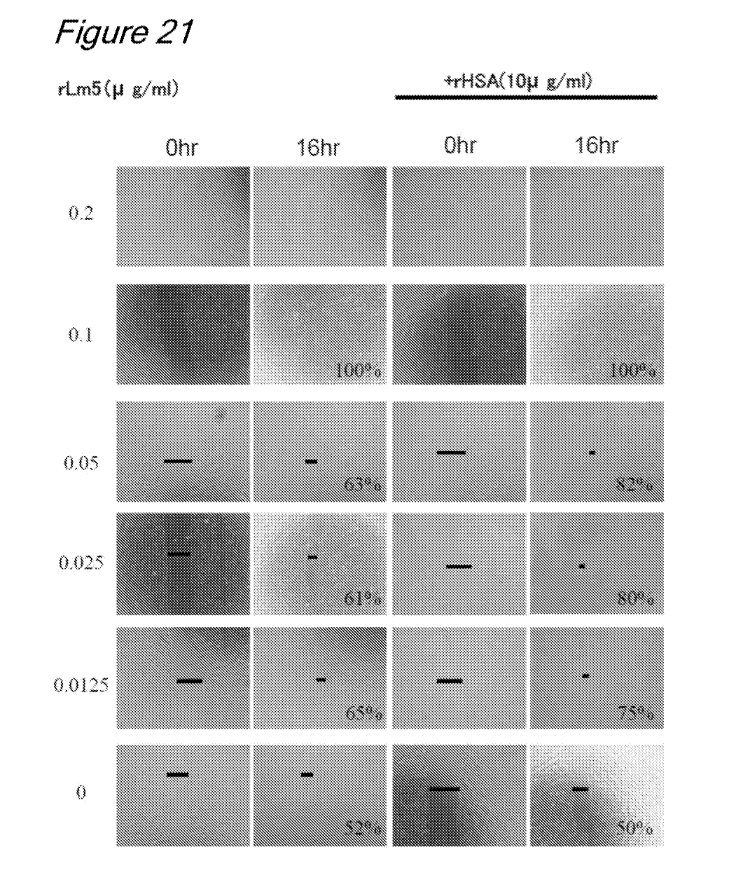

Cells were seeded to a 10% FBS culture at 160000 cells/well in a 24-well plate (Nunc), then they were cultured for 3 hours at 37° C., under an air layer of 5% CO₂ and 95% air. After culturing completed, a blue chip was used to put an injury of a specific width on the adhesion cell group, then the cells were washed twice in a serum-free medium, i.e., media wherein serum has been removed. The rLm5 that was concentration controlled in the serum-free medium was used to treat the wells at 37° C. for 1 hour. Treatment with rLm5 was conducted by mixing rHSA with rLm5 as necessary. After treatment with rLm5 completed, the treated surface was washed twice with serum-free medium, created by removing serum, and serum-free medium was added. The result was observed through a microscope, and the area around the wound was photographed. Then, the result was cultured for 16 hours at 37° C., under an air layer of 5% CO₂ and 95% air in a serum-free medium, and the same area was photographed (FIG. 21). The healing of the wound was assessed by measuring the width of the wound at the beginning and that at 16 hours therefrom to obtain the wound healing ratio.

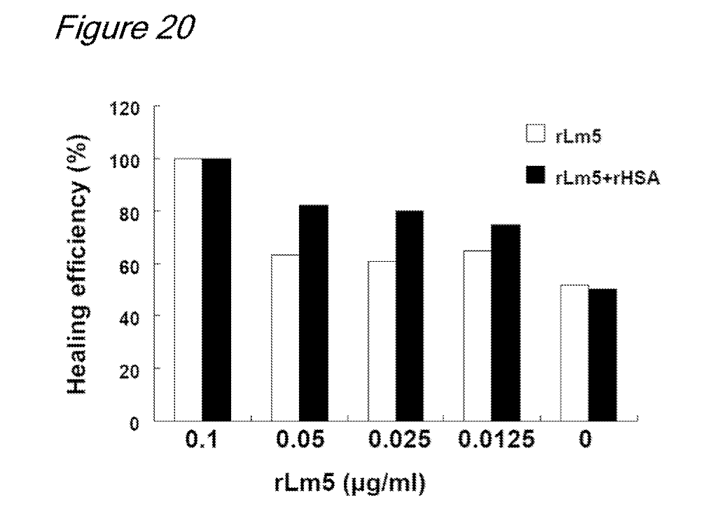

FIG. 20 shows the obtained wound healing ratio. A significant increase in the wound healing activity was exhibited when rHSA was added to Lm5.

The result indicates that an activity elevating effect of combining rHSA and other polypeptides for use is recognized not only for the cell adhesion activity and the cell scattering activity, but also for the wound healing activity previously reported as an activity of rLm5.

### Example 6

### Proliferation Assay Using Human Mesenchymal Stem Cells

This example shows the result of a proliferation assay using hMSC when different cell supporting materials are used.

Themedia used in the proliferation assay were medium with 5% Panexin (PAN-biotech) added to it instead of 10% FBS (P) and medium with 5% Panexin and 1 ng/ml of bFGF(Wako Pure Chemical) added to it (P+F). A maintenance medium (serum) comprising 10% FBS was also used for comparison. hMSC in each medium were seeded at 38400 cells/well in a 6 well plate (NUNC) treated with concentration-controlled extracellular matrix protein. They were cultured at 37° C., under an air layer of 5% CO₂ and 95% air for days, and the cells were collected on the 3^{rd} day, the 7^{th} day and the 10^{th} day from the beginning of culturing by enzyme treatment to count the number of cells using a hemacytometer.

The cell supporting material was prepared to form a mixture with 1 mg/ml of rLm5, 0.2 μg/ml of rLm5, 0.2 μg/ml of rLm5 and 10 μg/ml of rHSA added to it.

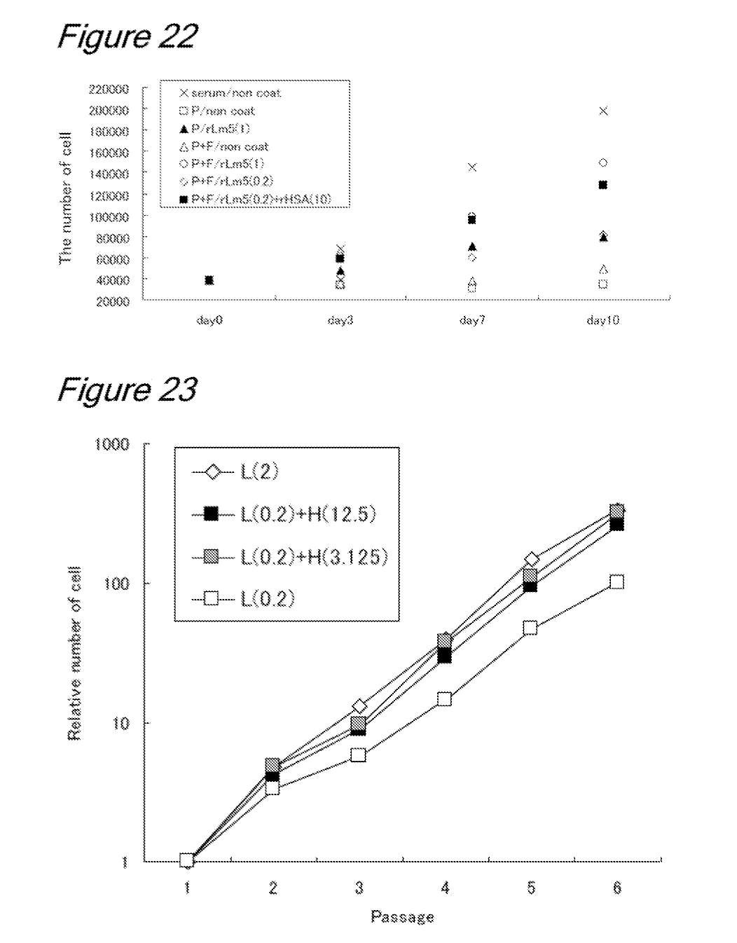

FIG. 22 shows the result of an investigation on the effect of hMSC on proliferation under different culture conditions. A significant increase in cell proliferation was seen when rHSA was added to 0.2 μg/ml of rLm5.

The above result indicates that an activity elevating effect of combining rHSA and other polypeptides for use is recognized not only for the cell adhesion activity, the cell scattering activity and the wound healing activity, but also for the proliferation stimulating activity for mesenchymal stem cells which is already reported as an activity of rLm5. Hence, it is presumed that rHSA and other polypeptides can increase all activities of rLm5 when they are used in combination with rLm5.

### Example 7

### Proliferation Assay Using EB3

This example shows result of a proliferation assay using EB3 when rLm5 is used as a cell supporting material.

The maintenance medium used for EB3 was medium (KSR-GMEM) with 10% Knockout™ Serum Replacement additives (KSR) (Invitrogen) instead of 10% FBS of the proliferation culture of Example 2. EB3 was seeded at 43000 cells/well to a 12 well plate (NUNC) treated with concentration-controlled extracellular matrix proteins of different types. They were cultured at 37° C., under an air layer of 5% CO₂ and 95% air for 2 days, then they were collected by enzyme treatment to count the number of cells using a hemacytometer.

EB3 was seeded again at 43000 cells/well to a 12 well plated treated with a concentration-controlled rLm5. The proliferation effects of rLm5 to EB3 at different conditions were compared by repeating the above process. The following variations of rLm5 were prepared: 2 μg/ml (L(2)), 0.2 μg/ml (L(0.2)), 0.2 μg/ml of rLm5 with 3.125 μg/ml of rHSA added to it (L(0.2)+H(3.125)), 0.2 μg/ml of rLm5 with 12.5 μg/ml of rHSA added to it (L(0.2)+H(12.5)).

FIG. 23 shows the result of a theoretical calculation showing by what factor EB3 will proliferate after 5 passages under the different culture conditions. According to the result shown in FIG. 23, the cell proliferation of the experiment using 0.2 μg/ml of rLm5 with rHSA added to it was about 3 times as much as the cell proliferation of only 0.2 μg/ml of rLm5, and equivalent to that of 2 μg/ml of rLm5.

The result shows that the effect of combination with rHSA is exhibited in the proliferation of mouse ES cells as well.

### Example 8

### Proliferation Assay Using EB3 and the Detection of an Undifferentiated State Markers

This example shows the result of a proliferation assay using EB3 and the detection result for an undifferentiated-state marker when different cell supporting materials are used.

The Example was performed with the same number of cells seeded in the proliferation assay of EB3 and the same passage interval as Example 7. The proliferation medium (S) used in Example 2 and KSR-GMEM (K) were used as the medium for the proliferation assay. The extracellular matrix proteins used for treating a 12 well plate were 1 mg/ml of Gl, 0.05 μg/ml of rLm5 (L0.05), and 0.05 μg/ml of rLm5 with 12.5 μg/ml of HSA added to it (L0.05+H12.5).

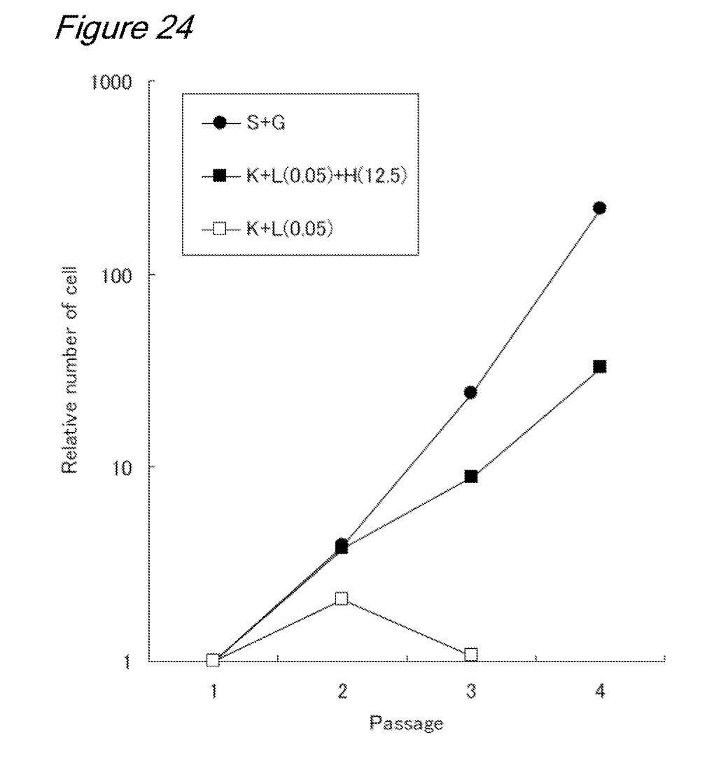

FIG. 24 shows the result of a theoretical calculation showing by what factor EB3 will proliferate after 3 passages under the given culture conditions. The result in FIG. 24 shows that proliferation terminates midway in an experiment using only 0.05 μg/ml of rLm5, but a substantial proliferation is exhibited in the experiment using 0.05 g/ml of rLm5 with rHSA added to it.

Next, S+G, K+L (0.05)+H (12.5) which exhibited proliferation were subjected to RT-PCR to detect undifferentiated-state markers, specifically to detect gene expression of Oct4, Sox2, Nanog, which are known in the art as undifferentiated-state markers of mouse ES cells. Note that under the condition of S+G, an experiment area maintained for the same period without LIF, which is known as the undifferentiated-state maintenance factor of mouse ES cells, was set as the negative control area (S(LIF−)+Gl).

TRIZOL (Invitrogen) was used to extract all RNAs from EB3 that has been cultured for 3 passages. After the extraction completed, reverse transcription was performed using ThermoScript RT-PCR System (Invitrogen) for cDNA synthesis. The primer of Table 2 was used to perform PCR using the synthesized cDNA as the mould.

TABLE 2
RT-PCR primer

Nanog

| | |
|---|---|
| 5′-AAGCAGAAGATGCGGACTGT-3′ | (SEQ ID NO: 7) |
| 5′-ACCACTGGTTTTTCTGCCAC-3′ | (SEQ ID NO: 8) |

Oct4

| | |
|---|---|
| 5′-TCTTTCCACCAGGCCCCCGGCTC-3′ | (SEQ ID NO: 9) |
| 5′-TGCGGGCGGACATGGGGAGATCC-3′ | (SEQ ID NO: 10) |

Sox2

| | |
|---|---|
| 5′-TAGAGCTAGACTCCGGGCGATGA-3′ | (SEQ ID NO: 11) |
| 5′-TTGCCTTAAACAAGACCACGAAA-3′ | (SEQ ID NO: 12) |

Gapdh

| | |
|---|---|
| 5′-CACCATGGAGAAGGCCGGGG-3′ | (SEQ ID NO: 13) |
| 5′-GACGGACACATTGGGGGTAG-3′ | (SEQ ID NO: 14) |

The degeneration reaction of the genes were performed at 94° C., for 30 seconds, the annealing reaction was performed for 30 seconds, and the extension reaction was performed at 72° C., for 20 seconds. The annealing reaction was performed at 61° C. for Oct4, and at 54° C. for Sox2, and Nanog.

FIG. 25 shows the detection result of the undifferentiated-state markers using RT-PCR. The result of FIG. 25 shows that 3 undifferentiated-state markers are expressed for K+L(0.05)+H(12.5) similar to S+Gl. This result suggests that EB3 maintains its undifferentiated state even when rHSA is combined for use with rLm5.

The result shows that the combination of rLm5 and rHSA not only has advantageous effects in proliferation, but it also maintains the proper undifferentiated-state of mouse ES cells for the retained mouse ES cells.

### Sequence Listing Free Text

### <SEQ ID NO: 1>

SEQ ID NO: 1 shows the nucleotide sequence of human laminin α3 chain.

### <SEQ ID NO: 2>

SEQ ID NO: 2 shows the amino acid sequence of human laminin α3 chain.

### <SEQ ID NO: 3>

SEQ ID NO: 3 shows the nucleotide sequence of human laminin β3 chain.

### <SEQ ID NO: 4>

SEQ ID NO: 4 shows the amino acid sequence of human laminin β3 chain.

### <SEQ ID NO: 5>

SEQ ID NO: 5 shows the nucleotide sequence of human laminin γ2 chain.

### <SEQ ID NO: 6>

SEQ ID NO: 6 shows the amino acid sequence of human laminin γ2 chain.

### <SEQ ID NOs: 7 to 14>

SEQ ID NOs: 7 to 14 show the nucleotide sequences of RT-PCR primers used for undifferentiated-state marker detection in EB3 cells.

1. A method for culturing cells in a system containing laminin-5 characterized in that the cells are cultured with a polypeptide selected from a group consisting of: a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone, under a condition wherein laminin-5 is immobilized.

2. The method according to claim 1, wherein the protein belonging to the tumor necrosis factor (TNF) family is a receptor activator NFₖB ligand (RANKL).

3. The method according to claim 1, wherein peptone is selected from a group consisting of a cotton seed derived peptone, a soy bean derived peptone, a wheat derived peptone and a pea derived peptone.

4. The method according to claim 1, wherein a cell culture vessel is immobilized with laminin-5 after it is immobilized with polypeptide, or the vessel is immobilized with polypeptide and laminin at the same time.

5. The method according to claim 1, wherein an activity of laminin-5 against the cells, selected from a group consisting of a cell adhesion activity, a cell scattering activity, a wound healing activity, a proliferation stimulating activity, an activity for maintaining undifferentiated-state and an activity for maintaining pluripotency, is increased.

6. The method according to claim 1, wherein the cells are selected from a group consisting of pluripotent stem cells, tissue stem cells, somatic cells, germ cells and sacroma cells.

7. The method according to claim 6, wherein the pluripotent stem cells are selected from embryonic stem cells, induced pluripotent stem cells, embryonic germ cells or germline stem cells;
the tissue stem cells are selected from mesenchymal stem cells, hepatic stem cells, pancreatic stem cells, neural stem cells, skin stem cells or hematopoietic stem cells; or
the somatic cells are selected from hepatic cells, pancreatic cells, muscle cells, osteocytes, osteoblasts, osteoclasts, cartilage cells, fat cells, skin cells, fibroblasts, pancreatic cells, kidney cells, pneumocytes or blood cells, which are lymphocites, red blood cells, white blood cells, monocytes, macrophage or megakaryocytes.

8. The method according to claim 1, wherein the cells are derived from a species selected from a group consisting of a mouse, a rat or a human.

9. The method according to claim 1, wherein the polypeptide is used at a concentration of 1 μg/ml to 200 μg/ml.

10. The method according to claim 1, wherein the polypeptide is used at a concentration of 3.125 μg/ml to 12.5 μg/ml.

11. The method according to claim 1, wherein two or more types of polypeptides are immobilized.

12. A composition to be used for immobilizing a cell culture vessel for culturing cells in a system comprising laminin-5, wherein the composition comprises a polypeptide selected from a group consisting of: a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone.

13. The composition according to claim 12, which further comprises laminin-5.

14. A kit for use in a method for culturing cells in a system comprising laminin-5, wherein the kit is immobilized with a polypeptide selected from a group consisting of: a protein in blood other than extracellular matrix proteins, which is, serum, serum albumin, prealbumin, immunoglobulin, α-globulin, β-globulin, α1-antitrypsin (α1-AT), heptoglobin (Hp), α2-macroglobulin (α2-M), α-fetoprotein (AFP), transferrin, retinol-binding protein (RBP) or adiponectin; gelatin; a protein belonging to a tumor necrosis factor (TNF) family; and peptone.

15. The kit according to claim 14 which is further immobilized with laminin-5.
